(19) United States
(12) Patent Application Publication
(10) Pub. No.: US 2026/0355271 A1
(43) Pub. Date: Nov. 12, 2026

(54) DIETARY SUPPLEMENT AND METHODS OF USE THEREOF

(71) Applicant: MAPLE MOUNTAIN GROUP, INC., Springville, UT (US)

(72) Inventors: Asma Ishaq, Newport Beach, CA (US); Jennifer Anderson, Springville, UT (US); Grace Ashton, Springville, UT (US)

(21) Appl. No.: 19/137,123

(22) PCT Filed: Dec. 11, 2023
(86) PCT No.: PCT/US2023/083383

§ 371 Date: Jun. 9, 2025

Related U.S. Application Data
(60) Provisional application No. 63/431,425, filed on Dec. 9, 2022.
(60) Provisional application No. 63/440,335, filed on Jan. 20, 2023.

(51) Int. Cl. A61K 36/61; A61K 9/48; A61K 31/122; A61K 31/164; A61K 31/352; A61K 31/455; A61K 31/522; A61K 31/7004; A61K 33/00; A61K 33/08; A61K 33/26; A61K 33/30; A61K 33/34; A61K 36/232; A61K 36/28; A61K 36/31; A61K 36/42; A61K 36/45; A61K 36/53; A61K 36/534; A61K 36/537; A61K 36/67; A61K 36/73; A61K 36/88; A61K 36/9068; A61K 45/06

(52) U.S. Cl. CPC A61K 36/61; A61K 9/485; A61K 9/4858; A61K 9/4866; A61K 31/122; A61K 31/164; A61K 31/352; A61K 31/455; A61K 31/522; A61K 31/7004; A61K 33/00; A61K 33/08; A61K 33/26; A61K 33/30; A61K 33/34; A61K 36/232; A61K 36/28; A61K 36/31; A61K 36/42; A61K 36/45; A61K 36/53; A61K 36/534; A61K 36/537; A61K 36/67; A61K 36/73; A61K 36/88; A61K 36/9068; A61K 45/06

(57) ABSTRACT

The invention is directed to dietary supplements to supplement a woman's specific nutritional needs during the menstrual cycle.

This application claims priority to U.S. Provisional Application No. 63/431,425 filed on Dec. 9, 2022 and U.S. Provisional Application No. 63/440,335 filed on Jan. 20, 2023, the entire contents of which are incorporated herein by reference.

### FIELD OF THE INVENTION

The present invention is directed to dietary supplements. In embodiments, it is directed to compositions, combination products or kits and methods to supplement a woman's specific nutritional needs during the menstrual cycle.

### BACKGROUND

The menstrual cycle is a sequence of periodic physiological changes that occurs in the female reproductive system (specifically in the uterus and ovaries) and that makes pregnancy possible. The menstrual cycle of a woman has an average duration of about 28 days and starts on the first day of the period and ends the day before the following period begins. The four phases of the menstrual cycle are menstruation, the follicular phase, ovulation and the luteal phase.

### SUMMARY OF THE INVENTION

In a first aspect, the present invention provides a method of treating or alleviating at least one menstrual cycle related symptom, or a method of supporting women during the menstrual cycle. comprising the oral administration of one. two or all. preferably all, of:
(i) a first composition (a) comprising clove and immortelle;
(ii) a second composition (b) comprising theacrine and broccoli; and
(iii) a third composition (c) comprising palmitoylethanolamide and ancient peat.

In a second aspect, the present invention provides a method of treating or alleviating at least one menstrual cycle related symptom, comprising the oral administration of one, two or all, preferably all, of:
(i) a first composition (a) comprising clove, immortelle, CoQ10, peppermint, ginger, a source, form or compound of copper, Dong quai, cranberry, sage, niacinamide, a source, form or compound of magnesium and a source. form or compound of potassium;
(ii) a second composition (b) comprising theacrine, a source, form or compound of iron, a source, form or compound of iodine, maca, inositol, broccoli and quercetin; and
(iii) a third composition (c) comprising palmitoylethanolamide, ancient peat, apple, saffron, chasteberry, a source, form or compound of zinc, black pepper, ginger and melon.

In a third aspect, the present invention provides a method of supporting women during the menstrual cycle, comprising the oral administration of one, two or all, preferably all, of:
(i) a first composition (a) comprising clove, immortelle, CoQ10, peppermint, ginger, a source, form or compound of copper, Dong quai, cranberry, sage, niacinamide, a source, form or compound of magnesium and a source, form or compound of potassium;
(ii) a second composition (b) comprising theacrine, a source, form or compound of iron, a source, form or compound of iodine, maca, inositol, broccoli and quercetin; and
(iii) a third composition (c) comprising palmitoylethanolamide, ancient peat, apple, saffron, chasteberry. a source, form or compound of zinc, black pepper, ginger and melon.

In aspects described herein, the first composition (a), the second composition (b) and the third composition (c) can be administered separately. The first composition (a), the second composition (b) and the third composition (c) can additionally be administered consecutively during the menstrual cycle.

In a fourth aspect, the present invention provides a combination product or kit comprising one, two or all, preferably all, of:
(i) a first composition (a) comprising clove and immortelle;
(ii) a second composition (b) comprising theacrine and broccoli; and
(iii) a third composition (c) comprising palmitoylethanolamide (PEA) and ancient peat.

In a fifth aspect, the present invention provides a combination product or kit comprising one, two or all, preferably all, of:
(i) a first composition (a) comprising clove, immortelle, CoQ10, peppermint, ginger, a source, form or compound of copper, Dong quai, cranberry, sage, niacinamide, a source, form or compound of magnesium and a source. form or compound of potassium;
(ii) a second composition (b) comprising theacrine, a source, form or compound of iron, a source, form or compound of iodine, maca, inositol, broccoli and quercetin; and
(iii) a third composition (c) comprising palmitoylethanolamide (PEA), ancient peat, apple, saffron, chasteberry, a source, form or compound of zinc, black pepper, ginger and melon.

In a sixth aspect, the present invention provides a combination product or kit according to the fourth and fifth aspects of the invention for use in the alleviation or treatment of at least one menstrual cycle related symptom or in a method of supporting women during the menstrual cycle.

In one embodiment, the combination product or kit according to the fourth and fifth aspects of the invention is for use in the alleviation of treatment of hormone imbalance, hormonal decline, premenstrual tension and premenstrual syndrome (PMS).

In a seventh aspect, the present invention provides a use of the combination product or kit according to the fourth and fifth aspects of the invention for improving or lessening alterations of mood, energy levels, mood swings, mild menstrual aching, or other mild discomforts associated with the menstrual cycle.

In an eighth aspect, the present invention provides a use of the combination product or kit according to the fourth and fifth aspects of the invention as a dietary or nutritional supplement.

In a ninth aspect, the present invention provides a composition (a) comprising clove, immortelle and optionally one or more ingredients selected from CoQ10, peppermint, ginger, a source, form or compound of copper, Dong quai, cranberry, sage, niacinamide, a source, form and compound of magnesium and a source, form or compound of potassium.

In a tenth aspect, the present invention provides a composition (a) comprising clove, immortelle, CoQ10, peppermint, ginger, a source, form or compound of copper, Dong quai, cranberry, sage, niacinamide, a source. form or compound of magnesium and a source, form or compound of potassium.

In an eleventh aspect, the present invention provides a composition (a) according to the ninth and tenth aspect of the invention for use in the alleviation or treatment of symptoms associated with the menstruation phase of the menstrual cycle.

In a twelfth aspect, the present invention provides a pharmaceutical composition or a dietary or a nutritional supplement comprising the composition (a) according to the ninth and tenth aspect of the invention.

In a thirteenth aspect, the present invention provides a method of alleviating or treating menstrual cycle related symptoms or a method of supporting women during the menstrual cycle comprising administering composition (a) according to the ninth and tenth aspect of the invention.

In a fourteenth aspect, the present invention provides a composition (b) comprising theacrine, and broccoli and optionally one or more ingredients selected from a source, form or compound of iron, a source, form or compound of iodine, maca, inositol and quercetin.

In a fifteenth aspect, the present invention provides a composition (b) comprising theacrine, a source, form or compound of iron, a source. form or compound of iodine, maca, inositol, broccoli and quercetin.

In a sixteenth aspect, the present invention provides a composition (b) according to the fourteenth and fifteenth aspects of the invention for use in the alleviation or treatment of symptoms associated with the follicular phase of the menstrual cycle from the conclusion of menses or menstruation phase.

In a seventeenth aspect, the present invention provides a pharmaceutical composition or a dietary or a nutritional supplement comprising the composition (b) according to the fourteenth and fifteenth aspects of the invention.

In an eighteenth aspect, the present invention provides a method of alleviating or treating menstrual cycle related symptoms or a method of supporting women during the menstrual cycle comprising administering composition (b) according to the fourteenth and fifteenth aspect of the invention.

In a nineteenth aspect, the present invention provides a composition (c) comprising palmitoylethanolamide (PEA), ancient peat and optionally one or more ingredients selected from apple, saffron, chasteberry, a source, form or compound of zinc, black pepper, ginger and melon

In a twentieth aspect, the present invention provides a composition (c) comprising palmitoylethanolamide (PEA), ancient peat, apple, saffron, chasteberry, a source, form or compound of zinc, black pepper, ginger and melon.

In a twenty first aspect, the present invention provides a composition (c) according to the nineteenth and twentieth aspects of the invention for use in the alleviation or treatment of symptoms associated with the luteal phase of the menstrual cycle.

In a twenty second aspect, the present invention provides a pharmaceutical composition or a dietary or a nutritional supplement comprising composition (c) according to the nineteenth and twentieth aspects of the invention.

In a twenty third aspect, the present invention provides a method of alleviating or treating menstrual cycle related symptoms or a method of supporting women during the menstrual cycle comprising administering composition (c) according to the nineteenth and twentieth aspects of the invention.

In a twenty fourth aspect, the present invention provides a dietary or nutritional supplement comprising the composition (a) according to the ninth and tenth aspect aspects of the invention, and/or the composition (b) according to the fourteenth and fifteenth aspect of the invention and/or the composition (c) according to the nineteenth and twentieth aspects of the invention.

### BRIEF DESCRIPTION OF THE DRAWINGS

FIG. 1 illustrates a blister pack comprising the combination product or kit of the present invention.

### DETAILED DESCRIPTION

Dietary supplements are commonly administered to treat specific medical conditions or as general nutritional supplements. Nutrition plays a critical role in maintaining good health, especially in women during child-bearing years.

Every month the fluctuation of hormones accompanying the female menstrual cycle may result in physical and emotional changes that can affect how a woman is feeling.

It has been demonstrated that diet, stress and environmental toxins can cause perturbations in the rhythmicity of the metabolism and hormones that drive the menstrual cycle. The loss of this metabolic and hormone rhythmicity can be associated with conditions such as premenstrual syndrome (PMS) and premenstrual dysphoric disorder (PMDD).

Therapeutic strategies, such as dietary changes, can assist restoration of metabolic and hormone rhythmicity (Scientific Reports, volume 8, Article number: 14568 (2018)).

The American College of Obstetricians and Gynecologists recommends exercise and dietary changes (e.g. complex carbohydrates and calcium rich foods), calcium supplementation and improved sleep for mild to moderate menstrual related symptoms. Pharmacological solutions for severe symptoms (hormonal contraceptives, diuretics, NSAIDS) mostly address somatic aspects, while anti-depressants, dopamine agonists. anxiolytics, and other psychoactive drugs are prescribed for behavioural/mood symptoms. However, the pharmacologically active agents currently used to treat menstrual cycle-induced symptoms are less than ideal, being costly and having significant side effects.

Thus, there is a need for nutritional or supplement products and methods that can support and meet the unique nutritional needs of each phase of the menstrual cycle.

The present invention provides methods, products and compositions for supporting women throughout the menstrual cycle by providing nutrients needed at specific times in the menstrual cycle.

Therefore the methods, products and compositions of the present invention provide specific nutritional support that helps with the metabolic, nutritional and hormone fluctuations occurring in the menstrual cycle, promoting good health and assisting with the nutritional needs associated with each phase of the menstrual cycle.

The present invention also provides a treatment of menstrual cycle related symptoms.

The methods, compositions and products of the present invention follow the user through their menstrual cycle and provide support based on the unique needs of each phase of the menstrual cycle. The methods, compositions and products of the present invention are customizable depending on cycle length. Each of the three compositions of the methods and products of the present invention matches up with a different phase of the menstrual cycle, so it optimizes hormonal and overall health and can help lessen or alleviate alterations of mood, low energy levels, occasional mood swings, mild menstrual aching, or other mild discomforts associated with the menstrual cycle.

These and other objects, which will be clear from the detailed description that follows, are attained by the methods, compositions and products of the present invention due to the technical characteristics claimed in the attached claims.

As used herein, the phrase “menstrual cycle” refers to the monthly cycle of changes in the ovaries and the lining of the uterus (endometrium). The four phases of the menstrual cycle are menstruation phase (menses), follicular phase, ovulation and the luteal phase.

The menstruation phase is the elimination of the thickened lining of the uterus (endometrium) from the body. Average length of menstruation (menses) is between about three days and about one week. In some embodiments, menstruation can be fewer than about three days or longer than about one week, up to about two weeks or more.

The follicular phase begins with onset of menses and ends with ovulation, and is characterized by release of hormones stimulating follicle production and endometrial thickening.

The average follicular phase can last for about 16 days, but it can range from about 11 to about 27 days, depending on the menstrual cycle. The follicular phase can make up the first half of the menstrual cycle.

Ovulation is the rupture of the follicle and release of a mature egg from the ovarian surface, stimulated by Luteinizing Hormone (LH). Ovulation can occur at mid-cycle, or about day 14 of a 28 days menstrual cycle, although this can vary among individuals. Ovulation can last about 24 hours. The released egg is transported to the uterus in preparation for fertilization.

Luteal phase: During ovulation, the egg is released from the follicle, but the ruptured follicle transforms into a glandular structure (corpus luteum) secreting progesterone and estrogen, for maintenance of the endometrium in anticipation of fertilization and implantation. Unless fertilization occurs, the cycle ends with the corpus luteum withering, which can be around about day 22 of a 28 day cycle. The drop in progesterone then causes shedding of part of the endometrium, which is menstruation (menses).

The luteal phase can last from about 11 to about 17 days, being the average length of about 14 days.

Although there exists variance among individuals, a typical menstrual cycle in human females is about 28 days. However, it is recognised that the menstrual cycle can differ in length from woman to woman and also in any one woman can be irregular, some months being about 28 days, other months being fewer than about 28 days from onset of menses to onset of menses, and/or in other months being greater than about 28 days from the onset of menses to onset of menses.

The present invention provides a method of treating or alleviating at least one menstrual cycle related symptom, comprising the oral administration of one, two or all, preferably all, of:
(i) a first composition (a) comprising clove and immortelle;
(ii) a second composition (b) comprising theacrine and broccoli; and
(iii) a third composition (c) comprising palmitoylethanolamide and ancient peat.

The present invention provides a method of alleviating at least one menstrual cycle related symptom, comprising the oral administration of one, two or all, preferably all, of:
(i) a first composition (a) comprising clove and immortelle;
(ii) a second composition (b) comprising theacrine and broccoli; and
(iii) a third composition (c) comprising palmitoylethanolamide and ancient peat.

The present invention provides a method of supporting women during the menstrual cycle, comprising the oral administration of one, two or all, preferably all, of:
(i) a first composition (a) comprising clove and immortelle;
(ii) a second composition (b) comprising theacrine and broccoli; and
(iii) a third composition (c) comprising palmitoylethanolamide and ancient peat.

The first composition (a) of the methods of the present invention can further comprise CoQ10, peppermint, ginger, a source, form or compound of copper, cranberry, sage, niacinamide, a source, form or compound of magnesium and a source, form or compound of potassium, Dong quai, or a combination thereof.

The second composition (b) of the methods of the present invention can further comprise a source, form or compound of iron, a source, form or compound of iodine, maca, inositol, quercetin or a combination thereof.

The third composition (c) of the methods of the present invention can further comprise apple, saffron, chasteberry, a source, form or compound of zinc, black pepper, ginger. melon or a combination thereof.

In one embodiment, the methods of the present invention, comprise the oral administration of one, two or all, preferably all, of:
(i) a dosage form including the first composition (a) comprising:
   about 100 mg to about 300 mg of clove, and
   about 100 mg to about 300 mg of immortelle;
(ii) a dosage form including the second composition (b) comprising:
   about 1 mg to about 500 mg of theacrine and
   about 1 mg to about 300 mg of broccoli; and
(iii) a dosage form including the third composition (c) comprising
   about 150 mg to about 500 mg of palmitoylethanolamide (PEA), and
   about 10 mg to about 300 mg of ancient peat.

The present invention further provides a method of treating or alleviating at least one menstrual cycle related symptom, comprising the oral administration of one, two or all, preferably all, of:
(i) a first composition (a) comprising clove, immortelle (also called Everlasting), CoQ10, peppermint, ginger, a source, form or compound of copper, Dong quai, cranberry, sage, niacinamide, a source, form or compound of magnesium and a source, form or compound of potassium;
(ii) a second composition (b) comprising theacrine, a source, form or compound of iron, a source, form or compound of iodine, maca, inositol, broccoli and quercetin; and
(iii) a third composition (c) comprising palmitoylethanolamide, ancient peat, apple, saffron, chasteberry, a source, form or compound of zinc, black pepper, ginger and melon.

The present invention provides a method of supporting women during the menstrual cycle, comprising the oral administration of one, two or all, preferably all, of:
(i) a first composition (a) comprising clove, immortelle, CoQ10, peppermint, ginger, a source, form or compound of copper, Dong quai, cranberry, sage, niacinamide, a source, form or compound of magnesium and a source, form or compound of potassium;
(ii) a second composition (b) comprising theacrine, a source, form or compound of iron, a source, form or compound of iodine, maca, inositol, broccoli and quercetin; and
(iii) a third composition (c) comprising palmitoylethanolamide, ancient peat. apple. saffron, chasteberry, a source, form or compound of zinc, black pepper, ginger and melon.

The fruit, herbs, plants, roots and vegetables as used in the compositions and methods described herein can be used in the form of an extract thereof, a power thereof, or a derivative thereof.

In one embodiment of the methods described herein, the first composition (a) comprises:
clove extract or clove flower,
immortelle extract or immortelle flower,
CoQ10.
peppermint extract,
ginger root extract,
a source, form or compound of copper selected from copper gluconate, copper sulfate, copper oxide and combinations thereof,
Dong quai extract,
cranberry extract,
sage extract or sage aerial parts,
niacinamide,
magnesium oxide, and
a source, form or compound of potassium selected from potassium phosphate, tripotassium phosphate, potassium sorbate and combinations thereof.

In one embodiment of the methods described herein, the second composition (b) comprises:
Theacrine,
a source, form or compound of iron selected from ferrous bisglycinate chelate, iron sulfate and combinations thereof
potassium iodide,
maca extract or maca root,
inositol,
broccoli sprout powder, broccoli crown or broccoli extract. and
quercetin.

In an embodiment of the methods described herein, the third composition (c) comprises:
palmitoylethanolamide,
ancient peat extract,
apple fruit extract,
saffron extract,
chasteberry extract,
a source, form or compound of zinc selected from zinc gluconate, zinc oxide, and combinations thereof,
black pepper extract,
ginger root extract, and
melon concentrated.

In one embodiment the methods of the present invention, comprise the oral administration of one, two or all, preferably all, of:
(i) a dosage form including the first composition (a) comprising:
   about 100 mg to about 300 mg of clove,
   about 100 mg to about 300 mg of immortelle,
   about 1 mg to about 50 mg of CoQ10,
   about 1 mg to about 80 mg of peppermint,
   about 1 mg to about 80 mg of ginger,
   about 1 mg to about 100 mg of a source, form or compound of magnesium,
   about 1 mg to about 100 mg of a source, form or compound of potassium,
   about 0.01 to about 20 mg of a source, form or compound of copper,
   about 1 mg to about 100 mg of Dong quai,
   about 1 mg to about 90 mg of cranberry,
   about 1 mg to about 80 mg of sage, and
   about 1 mg to about 50 mg of niacinamide;
(ii) a dosage form including the second composition (b) comprising:
   about 1 mg to about 500 mg of theacrine
   about 0.5 mg to about 50 mg of a source, form or compound of iron,
   about 0.005 mg to about 0.5 mg a source, form or compound of iodine,
   about 1 mg to about 300 mg of maca,
   about 5 mg to about 300 mg of inositol,
   about 1 mg to about 300 mg of broccoli, and
   about 1 mg to about 200 mg of quercetin; and
(iii) a dosage form including the third composition (c) comprising
   about 150 mg to about 500 mg of palmitoylethanolamide (PEA),
   about 10 mg to about 300 mg of ancient peat,
   about 1 mg to about 300 mg apple,
   about 1 mg to about 100 mg of saffron,
   about 1 mg to about 100 mg of chasteberry,
   about 1 mg to about 150 mg of a source, form or compound of zinc,
   about 0.5 mg to about 50 mg of black pepper,
   about 1 mg to about 80 mg of ginger, and
   about 0.5 mg to about 100 mg of melon or an extract thereof.

In one embodiment the methods of the present invention, comprise the oral administration of one, two or all, preferably all, of:
(i) a dosage form including the first composition (a) comprising:
   about 100 mg to about 300 mg of clove or an extract thereof,
   about 100 mg to about 300 mg of immortelle or an extract thereof,
   about 1 mg to about 50 mg of CoQ10,
   about 1 mg to about 80 mg of peppermint or an extract thereof,
   about 1 mg to about 80 mg of ginger or an extract thereof,
   about 1 mg to about 100 mg of magnesium oxide,
   about 1 mg to about 100 mg of potassium phosphate,
   about 0.01 to about 20 mg of copper gluconate,
   about 1 mg to about 100 mg of Dong quai or an extract thereof,
   about 1 mg to about 90 mg of cranberry or an extract thereof,
   about 1 mg to about 80 mg of sage or an extract thereof, and
   about 1 mg to about 50 mg of niacinamide;
(ii) a dosage form including the second composition (b) comprising:
   about 1 mg to about 500 mg of theacrine
   about 0.5 mg to about 50 mg of ferrous bisglycinate chelate,
   about 0.005 mg to about 0.5 mg potassium iodide,
   about 1 mg to about 300 mg of maca or an extract thereof,
   about 5 mg to about 300 mg of inositol,
   about 1 mg to about 300 mg of broccoli or an extract thereof, and
   about 1 mg to about 200 mg of quercetin; and
(iii) a dosage form including the third composition (c) comprising
   about 150 mg to about 500 mg of palmitoylethanolamide (PEA)
   about 10 mg to about 300 mg of ancient peat or an extract thereof,
   about 1 mg to about 300 mg of apple or an extract thereof,
   about 1 mg to about 100 mg of saffron or an extract thereof,
   about 1 mg to about 100 mg of chasteberry or an extract thereof,
   about 1 mg to about 150 mg of zinc gluconate or zinc oxide or combination,
   about 0.5 mg to about 50 mg of black pepper or an extract thereof,
   about 1 mg to about 80 mg of ginger or an extract thereof, and
   about 0.5 mg to about 100 mg of melon or an extract thereof.

The methods of the present invention can comprise the oral administration of the first composition (a). the second composition (b) and the third composition as described herein.

In the methods as described herein, compositions (a), (b) and (c) can be administered separately. Two or all of compositions (a), (b) and (c) can be administered consecutively. In one embodiment two or all, preferable all, of compositions (a), (b) and (c) can be administered separately and consecutively during the menstrual cycle.

In one embodiment, compositions (a), (b) and (c) of the methods described herein are orally administered separately and consecutively during the menstrual cycle.

The first composition (a) and/or the second composition (b) and/or the third composition (c) of the methods of the present invention can further include inositol, lutein, L-carnitine, ascorbic acid, niacinamide, fennel, riboflavin, vitamin E, lycopene, methylcobalamin or combination thereof.

The methods of the present invention can comprise administering during a first fraction of the menstrual cycle the first composition (a), and/or administering during a second fraction of said menstrual cycle the second composition (b) and/or administering during a third fraction of said menstrual cycle the third composition (c).

The methods of the present invention can comprise administering during a first fraction of the menstrual cycle the first composition (a) and administering during a second fraction of said menstrual cycle the second composition (b) and administering during a third fraction of said menstrual cycle the third composition (c).

In one embodiment the first fraction of the menstrual cycle comprises at least one day of the menstruation phase.

In one embodiment the first fraction of the menstrual cycle comprises about 1 day, or about 2 days, or about 3 days, or about 4 days, or about 5 days, or about 6 days, or about 7 days or about 8 days of the menstruation phase. In one embodiment the first fraction of the menstrual cycle comprises about 4 days or about 5 days of the menstruation phase. In one embodiment the first fraction comprises the length of the menstruation phase.

In one embodiment, the first composition (a) can be administered for about 1 day, or for about 2 days, or for about 3 days, or for about 4 days, or for about 5 days, or for about 6 days, or for about 7 days or for about 8 days, starting administration the first day of the menstruation phase. The first composition (a) can be administered for between about 1 day and about 6 days, or for between about 1 day and about 5 days, or for between about 1 day and about 4 days, from the first day of the menstruation phase.

As used herein, the beginning of the menstrual cycle can refer to “day 1” of the menstrual cycle, and is the day of onset of menses or the first day of the menstruation phase.

In one embodiment the first fraction of the menstrual cycle comprises day 1 to day 8, or day 1 to day 7, or day 1 to day 6, or day 1 to day 5, or day 1 to day 4 of the menstrual cycle.

In one embodiment the second fraction of the menstrual cycle comprises at least one day of the follicular phase. Although the follicular phase starts on the first day of menstruation phase, in one embodiment of the present invention the second fraction of the menstrual cycle comprises at least one day of the follicular phase, excluding the menstruation phase, so from the conclusion of menses or menstruation phase.

In one embodiment the second fraction of the menstrual cycle comprises about 6 days, or about 7 days, or about 8 days, or about 9 days, or about 10 days, or about 11 days, or about 12 days, or about 13 days, or about 14 days, or about 15 days, or about 16 days. or about 17 days, or about 18 days of the follicular phase, from the conclusion of menses or menstruation phase. In one embodiment the second fraction of said menstrual cycle comprises between about 7 days and about 18 days, or between about 7 days and about 17 days, or between about 8 days and about 16 days of the follicular phase and from the conclusion of menses or menstruation phase.

In one embodiment, the second composition (b) can be administered for about 6 days, or for about 7 days, or for about 8 days, or for about 9 days, or for about 10 days, or for about 11 days, or for about 12 days, or for about 13 days, or for about 14 days, or for about 15 days, or for about 16 days, or for about 17 days, or for about 18 days, from the conclusion of menses or menstruation phase. In one embodiment, the second composition (b) can be administered for between about 6 days and about 18 days, or for between about 7 days and about 17 days, or for between about 8 and about 16 days, from the conclusion of menses or menstruation phase.

In one embodiment the second fraction of the menstrual cycle comprises day 4 to day 22, or day 4 to day 20, or day 4 to day 18, or day 4 to about day 16, or about day 5 to about day 23, or about day 5 to about day 20, or about day 5 to about day 18, or day 6 to day 24, or day 6 to day 22, or day 6 to day 20, or day 7 to day 25, or day 7 to day 23, or day 5 to day 21, or day 8 to day 26, or day 8 to day 18 of the menstrual cycle

In one embodiment the third fraction of said menstrual cycle comprises at least one day of the luteal phase. In one embodiment the third fraction of said menstrual cycle comprises at least one day from ovulation to onset of menses.

In one embodiment the third fraction of said menstrual cycle comprises about 8 days, or about 9 days, or about 10 days, or about 11 days, or about 12 days, or about 13 days, or about 14 days, or about 15 days, or about 16 days, or about 17 days, or about 18 days, or about 19 days, or about 20 days of the luteal phase.

In one embodiment, the third composition (c) is administered for about 8 days, or for about 9 days, or for about 10 days, or for about 11 days, or for about 12 days, or for about 13 days, or for about 14, or for about 15 days, or for about 16 days, or for about 17 days, or for about 18 days, or for about 19 days, or for about 20 days from the conclusion of the follicular phase. The third composition (c) can be administered for between about 8 days and about 20 days, or for between about 10 days and about 18 days, from the conclusion of the follicular phase

In one embodiment the second fraction of the menstrual cycle comprises day 12 to day 40, or day 12 to day 38 day, or day 13 to day 38, or day 14 to day 38, or day 13 to day 35, or day 12 to day 28, or day 13 to day 28 or day 14 to day 28 of the menstrual cycle.

In one embodiment the method of the invention comprises:
(i) daily administering the first composition (a) for about 3 to 8 days, or for about 4 to 5 days during a first fraction of the menstrual cycle;
(ii) daily administering the second composition (b) for about 7 to 18 days or for about 10 to 13 days during a second fraction of the menstrual cycle, and
(iii) daily administering the third composition (c) for about 8 to 20 days or for about 10 to 18 days during a third fraction of the menstrual cycle.

In one embodiment the first fraction of the menstrual cycle can start the first day of the menstruation phase. In one embodiment the second fraction of the menstrual cycle can start the first day after the conclusion of the menstruation phase. In one embodiment the third fraction of the menstrual cycle can start the first day of the luteal phase.

In one embodiment the first fraction of the menstrual cycle comprises from day 1 to day 4 of the menstrual cycle. In one embodiment the second fraction of the menstrual cycle comprises from about day 5 to about day 17 of the menstrual cycle. In one embodiment the third fraction of the menstrual cycle comprises from about day 18 to the last day of the menstrual cycle (e.g. from about day 18 to about day 35).

In one embodiment, the methods of the present invention can be used to treat at least one menstrual cycle related symptom. Examples of menstrual cycle related symptoms which can be treated include, but are not limited to hormone imbalance, hormonal decline, premenstrual tension and premenstrual syndrome (PMS).

The methods of the present invention can be used for improving or restoring hormone balance.

In one embodiment, the methods of the present invention can be used to treat at least one menstrual cycle symptoms and support women menstrual cycle. Examples of non-therapeutical menstrual cycle symptoms and supporting menstrual cycle can include, but are not limited to improving or lessening alterations of mood, energy levels, mood swings, mild menstrual aching, or other mild discomforts associated with the menstrual cycle.

In one embodiment, the methods of the present invention can be used to support women menstrual cycle and/or women's health.

In some embodiments, the methods can comprise administering to a subject a pharmaceutically effective amount of the first composition (a), the second composition (b) and the first composition (c) as described herein.

In one embodiment, the methods as described herein, further comprise administering one of the first composition (a), the second composition (b) or the third composition (c) about once per day.

The present invention provides a combination product or kit comprising one, two or all, preferably all, of:
(i) a dosage form including a first composition (a) comprising clove and immortelle;
(ii) a dosage form including a second composition (b) comprising theacrine and broccoli; and
(iii) a dosage form including the third composition (c) comprising palmitoylethanolamide and ancient peat.

The first composition (a) of the combination product or kit of the present invention may further comprise CoQ10, peppermint, ginger, a source, form or compound of copper, cranberry, sage, niacinamide, a source, form or compound of magnesium and a source, form or compound of potassium, Dong quai, or a combination thereof.

The second composition (b) of the combination product or kit of the present invention may further comprise a source, form or compound of iron, a source, form or compound of iodine, maca, inositol, quercetin or a combination thereof.

The third composition (c) of the combination product or kit of the present invention may further comprise apple, saffron, chasteberry, a source, form or compound of zinc, black pepper, ginger, melon or a combination thereof.

In one embodiment the combination product or kit of the present invention comprises one, two or all, preferably all, of:
(i) at least one dosage form including the first composition (a) comprising:
   about 100 mg to about 300 mg of clove, and
   about 100 mg to about 300 mg of immortelle;
(ii) at least one dosage form including the second composition (b) comprising:
   about 1 mg to about 500 mg of theacrine, and
   about 1 mg to about 300 mg of broccoli; and
(iii) at least one dosage form including the third composition (c) comprising
   about 150 mg to about 500 mg of palmitoylethanolamide (PEA), and
   about 10 mg to about 300 mg of ancient peat.

The present invention further provides a combination product or kit comprising one, two or all, preferably all, of:
(i) a dosage form including the first composition (a) comprising clove, immortelle, CoQ10, peppermint, ginger, a source, form or compound of copper, Dong quai, cranberry, sage, niacinamide, a source, form or compound of magnesium and a source, form or compound of potassium;
(ii) a dosage form including the second composition (b) comprising theacrine, a source, form or compound of iron, a source, form or compound of iodine, maca, inositol, broccoli and quercetin; and
(iii) a dosage form including the third composition (c) comprising palmitoylethanolamide (PEA), ancient peat, apple, saffron, chasteberry, a source, form or compound of zinc, black pepper, ginger and melon.

The fruit, herbs, plants, roots and vegetables as used in the compositions, dosage forms, combination products or kits described herein may be in the form of an extract thereof, a power thereof, or a derivative thereof.

In one embodiment the combination product or kit of the present invention comprises one, two or all. preferably all, of:
(i) at least one dosage form including the first composition (a) comprising:
   clove extract or clove flower,
   immortelle extract or immortelle flowering top,
   CoQ10,
   peppermint extract,
   ginger root extract,
   a source, form or compound of copper selected from copper gluconate, copper sulfate, copper oxide and combinations thereof,
   Dong quai,
   cranberry extract,
   sage extract,
   niacinamide,
   magnesium oxide, and
   a source, form or compound of potassium selected from potassium phosphate, tripotassium phosphate, potassium sorbate and combinations thereof;
(ii) at least one dosage form including the second composition (b) comprising:
   Theacrine,
   a source, form or compound of iron selected from ferrous bisglycinate chelate, iron sulfate and combinations thereof
   potassium iodide,
   maca root,
   inositol,
   broccoli sprout powder, broccoli crown or broccoli extract, and
   quercetin;
(iii) at least one dosage form including the third composition (c) comprising
   palmitoylethanolamide,
   ancient peat extract,
   apple extract.
   saffron extract,
   chasteberry extract,
   a source, form or compound of zinc selected from zinc gluconate, zinc oxide,
   and combinations thereof,
   black pepper extract.
   ginger root extract, and
   melon concentrate.

In one embodiment the combination product or kit of the present invention comprises one, two or all, preferably all, of:
(i) at least one dosage form including the first composition (a) comprising:
   about 100 mg to about 300 mg of clove,
   about 100 mg to about 300 mg of immortelle,
   about 1 mg to about 50 mg of CoQ10,
   about 1 mg to about 80 mg of peppermint,
   about 1 mg to about 80 mg of ginger,
   about 1 mg to about 100 mg of a source, form or compound of magnesium,
   about 1 mg to about 100 mg of a source, form or compound of potassium,
   about 0.01 to about 20 mg of a source, form or compound of copper.
   about 1 mg to about 100 mg of Dong quai,
   about 1 mg to about 90 mg of cranberry,
   about 1 mg to about 80 mg of sage, and
   about 1 mg to about 50 mg of niacinamide;
(ii) at least one dosage form including the second composition (b) comprising:
   about 1 mg to about 500 mg of theacrine
   about 0.5 mg to about 50 mg of a source, form or compound of iron,
   about 0.005 mg to about 0.5 mg a source, form or compound of iodine,
   about 1 mg to about 300 mg of maca,
   about 5 mg to about 300 mg of inositol,
   about 1 mg to about 300 mg of broccoli, and
   about 1 mg to about 200 mg of quercetin; and
(iii) at least one dosage form including the third composition (c) comprising:
   about 150 mg to about 500 mg of palmitoylethanolamide (PEA),
   about 10 mg to about 300 mg of ancient peat,
   about 1 mg to about 300 mg apple,
   about 1 mg to about 100 mg of saffron,
   about 1 mg to about 100 mg of chasteberry,
   about 1 mg to about 150 mg of a source, form or compound of zin.
   about 0.5 mg to about 50 mg of black pepper,
   about 1 mg to about 80 mg of ginger, and
   about 0.5 mg to about 100 mg of melon or an extract thereof.

In one embodiment the combination product or kit of the present invention comprises one, two or all, preferably all, of:
(i) at least one dosage form including the first composition (a) comprising:
   about 100 mg to about 300 mg of clove or an extract thereof,
   about 100 mg to about 300 mg of immortelle or an extract thereof,
   about 1 mg to about 50 mg of CoQ10,
   about 1 mg to about 80 mg of peppermint or an extract thereof,
   about 1 mg to about 80 mg of ginger or an extract thereof,
   about 1 mg to about 100 mg of magnesium oxide,
   about 1 mg to about 100 mg of potassium phosphate,
   about 0.01 to about 20 mg of copper gluconate,
   about 1 mg to about 100 mg of Dong quai or an extract thereof,
   about 1 mg to about 90 mg of cranberry or an extract thereof,
   about 1 mg to about 80 mg of sage or an extract thereof, and
   about 1 mg to about 50 mg of niacinamide;
(ii) at least one dosage form including the second composition (b) comprising:
   about 1 mg to about 500 mg of theacrine
   about 0.5 mg to about 50 mg of ferrous bisglycinate chelate,
   about 0.005 mg to about 0.5 mg potassium iodide,
   about 1 mg to about 300 mg of maca or an extract thereof,
   about 5 mg to about 300 mg of inositol,
   about 1 mg to about 300 mg of broccoli or an extract thereof, and
   about 1 mg to about 200 mg of quercetin; and
(iii) at least one dosage form including the third composition (c) comprising:
   about 150 mg to about 500 mg of palmitoylethanolamide (PEA)
   about 10 mg to about 300 mg of ancient peat or an extract thereof,
   about 1 mg to about 300 mg of apple or an extract thereof,
   about 1 mg to about 100 mg of saffron or an extract thereof,
   about 1 mg to about 100 mg of chasteberry or an extract thereof,
   about 1 mg to about 150 mg of a source, form or compound of zinc selected from zinc gluconate, zin oxide, or a combination thereof,
   about 0.5 mg to about 50 mg of black pepper extract,
   about 1 mg to about 80 mg of ginger or an extract thereof, and
   about 0.5 mg to about 100 mg of melon an extract thereof.

In one embodiment, the dosage forms including the first composition (a), the second composition (b), and the third composition (c) of the combination products or kits according to the present invention, are oral dosage forms or are orally administrable.

The first composition (a) and/or the second composition (b) and/or the third composition (c) of the combination product or kit of the present invention can further comprise inositol, lutein, L-carnitine, ascorbic acid, niacinamide, fennel, riboflavin, vitamin E, lycopene, methylcobalamin or combination thereof.

The combination products or kits of the present invention are for administration to a woman during a first, a second, and a third fraction of the menstrual cycle as described herein.

The combination products or kits of the present invention may be used for different cycle lengths.

The combination product or kit of the present invention may comprise from about 3 to about 8 dosage forms of the first composition (a), from about 7 to about 18 dosage forms of the second composition (b), and from about 10 to about 20 dosage forms of the third composition (c).

The combination products or kits of the present invention may be in the form of a blister pack.

The combination products or kits of the present invention may include instructions for use.

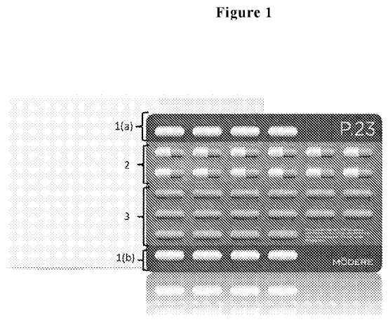

In one embodiment, as exemplified in FIG. 1, the combination products or kits of the present invention can comprise about 4 dosage forms of the first composition (a), about 12 dosage forms of the second composition (b), about 16 dosage forms of the third composition (c) and about four further dosage forms of the first composition (a), so the combination product or kit of the present invention can be used for different cycle lengths.

In one embodiment each dosage form of the first composition (a), the second composition (b) and the third composition (c) can be in the form of a pill. a tablet. a caplet, a tablet (such as a chewable tablet, a quick dissolve tablet or an effervescent tablet), a gummy, a capsule (e.g. hard gelatin capsule or a soft gelatin capsule), a gel cap. a powder, pellets, granules, beads, a liquid suspension or drinkable solutions, a food product, or any other form in the context of the present invention. One of ordinary skill in the art would recognize there are also other viable ways for delivering the nutritional supplement to a user. In embodiments, said oral form of the first composition (a), second composition (b) and third composition (c) may be in the form of a tablet, capsule, a gummy, granules, or a solution.

In one embodiment, the first composition (a), second composition (b) and third composition (c) of the combination products or kits of the present invention are in the form of tablets, capsules or gummies and are packaged in one package or blister pack. So the combination products or kits of the present invention may comprise from about 3 to about 8 tablets, capsules or gummies of the first composition (a), from about 7 to about 18 tablets, capsules or gummies of the second composition (b), and from about 10 to about 20 tablets, capsules or gummies of the third composition (c).

In one embodiment, the combination product or kit is for monthly use, wherein one of the first composition (a), or the second composition (b) or the third composition (c) are administered once per day.

The combination products or kits of the present invention may be used for the treatment of menstrual cycle-induced or related symptoms. The combination product or kit of the present application may be used for treating at least one menstrual cycle related symptom selected from the group consisting of, but are not limited to, hormone imbalance, hormonal decline, premenstrual tension and premenstrual syndrome (PMS).

The combination products or kits of the present invention may be used for improving or restoring hormone balance.

The present invention further provides use of the combination products or kits as described herein. For example, the combination product or kit of the present invention may be used for improving or lessening alterations of mood, energy levels, mood swings, mild menstrual aching, or other mild discomforts associated with the menstrual cycle.

The combination products or kits as described herein may be used as a women's health supplement.

The combination products or kits of the present invention may be used as a dietary or nutritional supplement.

The present invention also provides a dietary or nutritional supplement comprising the combination products or kits as described herein.

The present invention provides a composition (a) comprising clove, immortelle (also called Everlasting) and optionally one or more ingredients selected from: CoQ10, peppermint, ginger, a source, form or compound of copper, Dong quai, cranberry, sage, niacinamide, a source, form and compound of magnesium and a source, form or compound of potassium.

In one embodiment the composition (a) can comprise clove, immortelle (also called Everlasting), CoQ10, peppermint, ginger, a source, form or compound of copper, Dong quai, cranberry, sage, niacinamide, a source, form or compound of magnesium and a source, form or compound of potassium.

In one embodiment a dosage form including the composition (a) may comprise:
about 100 mg to about 300 mg of clove,
about 100 mg to about 300 mg of Immortelle, and
   optionally one or more ingredients selected from:
about 1 mg to about 50 mg of CoQ10,
about 1 mg to about 80 mg of peppermint,
about 1 mg to about 80 mg of ginger,
about 0.01 to about 20 mg of a source, form or compound of copper,
about 1 mg to about 100 mg of Dong quai,
about 1 mg to about 90 mg of cranberry,
about 1 mg to about 80 mg of sage,
about 1 mg to about 50 mg of niacinamide,
about 1 mg to about 100 mg of a source, form or compound of magnesium, and
about 1 mg to about 100 mg of a source, form or compound of potassium.

In one embodiment a dosage form including the composition (a) may comprise:
about 100 mg to about 300 mg of clove or an extract thereof,
about 100 mg to about 300 mg of immortelle or an extract thereof, and
   optionally one or more ingredients selected from:
about 1 mg to about 50 mg of CoQ10,
about 1 mg to about 80 mg of peppermint or an extract thereof,
about 1 mg to about 80 mg of ginger or an extract thereof,
about 0.01 to about 20 mg of a source, form or compound of copper selected from copper gluconate, copper sulfate, copper oxide and combinations thereof
about 1 mg to about 100 mg of Dong quai or an extract thereof,
about 1 mg to about 90 mg of cranberry or an extract thereof.
about 1 mg to about 80 mg of sage or an extract thereof,
about 1 mg to about 50 mg of niacinamide,
about 1 mg to about 100 mg of magnesium oxide, and
about 1 mg to about 100 mg of a source, form or compound of potassium selected from potassium phosphate, tripotassium phosphate, potassium sorbate or combinations thereof.

In one embodiment composition (a) is an oral composition or is orally administered.

In one embodiment composition (a) is for administration to a woman during the menstruation phase of the menstrual cycle.

In one embodiment composition (a) can be used for treating, improving or lessening symptoms associated with the menstruation phase of the menstrual cycle.

In one embodiment composition (a) can be used for the treatment of symptoms associated with the menstruation phase of the menstrual cycle. Composition (a) of the present application may be used for treating or alleviating symptoms associated with the menstruation phase of the menstrual cycle selected from the group consisting of, but are not limited to abdominal cramps, tiredness, bloating, tender breasts and mood swings.

Composition (a) of the present invention may help to relieve cramps, ease bloating, promote clearer looking skin, support energy levels, support iron metabolism, support liver health, support healthy blood flow, support urinary tract health, support antioxidant capacity.

Composition (a) of the present invention may help to support the liver and detoxification, reduce mild digestive discomforts, supports muscle function, support circulation. iron metabolism, energy levels and reduce mild discomfort during menstruation.

In one embodiment composition (a) may be used for lessening mild discomfort associated with menstruation.

In one embodiment composition (a) may be used as a women's health supplement.

In one embodiment composition (a) may be a dietary or nutritional supplement

In one embodiment the composition (a) may comprise:
clove extract or clove flower (e.g. clove flower bud),
immortelle extract or immortelle flower (for example immortelle flowering top), CoQ10,
peppermint extract (e.g. peppermint leaf extract),
ginger extract (e.g. ginger root extract),
a source, form or compound of copper selected from copper gluconate, copper
sulfate, copper oxide and combinations thereof,
Dong quai extract (e.g. Dong quai root extract).
cranberry extract,
sage extract (e.g. sage aerial parts extract),
niacinamide,
magnesium oxide, and
a source, form or compound of potassium selected from potassium phosphate, tripotassium phosphate, potassium sorbate or combinations thereof.

In one embodiment a dosage form including the composition (a) may comprise:
about 100 mg to about 300 mg of clove,
about 100 mg to about 300 mg of immortelle,
about 1 mg to about 50 mg of CoQ10,
about 1 mg to about 80 mg of peppermint,
about 1 mg to about 80 mg of ginger,
about 1 mg to about 100 mg of a source, form or compound of magnesium,
about 1 mg to about 100 mg of a source, form or compound of potassium,
about 0.01 to about 20 mg of a source, form or compound of copper,
about 1 mg to about 100 mg of Dong quai,
about 1 mg to about 90 mg of cranberry,
about 1 mg to about 80 mg of sage, and
about 1 mg to about 50 mg of niacinamide.

In one embodiment a dosage form including the composition (a) may comprise:
about 100 mg to about 300 mg of clove extract,
about 100 mg to about 300 mg of immortelle extract,
about 1 mg to about 50 mg of CoQ10,
about 1 mg to about 80 mg of peppermint extract,
about 1 mg to about 80 mg of ginger root extract,
about 1 mg to about 100 mg of magnesium oxide,
about 1 mg to about 100 mg of potassium phosphate,
about 0.01 to about 20 mg of copper gluconate,
about 1 mg to about 100 mg of Dong quai extract,
about 1 mg to about 90 mg of cranberry extract,
about 1 mg to about 80 mg of sage extract, and
about 1 mg to about 50 mg of niacinamide.

The present invention further provides a pharmaceutical composition or a dietary or nutritional supplement comprising composition (a) as described herein.

The present invention further provides a pharmaceutical composition comprising a pharmaceutically acceptable carrier, and an effective amount, for example a dosage form, of composition (a) as described herein.

The present invention further provides a dietary or nutritional supplement comprising a dietary or nutritionally acceptable carrier, and an effective amount, for example a dosage form, of composition (a) as described herein.

In one embodiment the composition (a), the dosage form including composition (a), and the pharmaceutical composition and dietary or nutritional supplement comprising composition (a) are for administration once per day.

The present invention further provides a method of treating menstrual cycle related symptoms or a method of supporting women during the menstrual cycle comprising composition (a) as described herein.

The present invention further provides a composition (b) comprising theacrine and broccoli, and optionally one or more ingredients selected from a source, form or compound of iron, quercetin, a source, form or compound of iodine, maca and inositol.

In one embodiment the composition (b) can comprise theacrine, a source, form or compound of iron, a source, form or compound of iodine, maca, inositol, broccoli and quercetin.

In one embodiment a dosage form including the composition (b) can comprise:
about 1 mg to about 500 mg of theacrine,
about 1 mg to about 300 mg of broccoli, and
   optionally one or more ingredients selected from:
about 0.5 mg to about 50 mg of a source, form or compound of iron,
about 0.005 mg to about 0.5 mg a source, form or compound of iodine,
about 1 mg to about 300 mg of maca,
about 5 mg to about 300 mg of inositol, and
about 1 mg to about 200 mg of quercetin.

In one embodiment a dosage form including the composition (b) can comprise:
about 1 mg to about 500 mg of theacrine,
about 1 mg to about 300 mg of broccoli or an extract thereof, and
   optionally one or more ingredients selected from:
about 0.5 mg to about 50 mg of a source, form or compound of iron selected from ferrous bisglycinate chelate, iron sulfate and combinations thereof.
about 0.005 mg to about 0.5 mg a source, form or compound of iodine,
about 1 mg to about 300 mg of maca or an extract thereof,
about 5 mg to about 300 mg of inositol, and
about 1 mg to about 200 mg of quercetin.

In one embodiment composition (b) is an oral composition or is orally administered.

In one embodiment composition (b) is for administration to a woman during the follicular phase of the menstrual cycle, from the conclusion of menses or menstruation phase.

In one embodiment composition (b) may be used for treating, improving or lessening symptoms associated with the follicular phase of the menstrual cycle, from the conclusion of menses or the menstruation phase.

In one embodiment composition (b) may be used for the treatment of symptoms associated with the follicular phase of the menstrual cycle, from the conclusion of menses or menstruation phase. Composition (b) of the present invention can be used for treating or alleviating symptoms associated with the follicular phase of the menstrual cycle, from the conclusion of menses or the menstruation phase, selected from the group consisting of, but are not limited to hormone imbalances, low libido, tiredness, tender breasts and mood swings.

In one embodiment composition (b) can be used for improving or restoring hormone balance, lessening breast tenderness or swelling, improving energy levels, mood and healthy libido.

Composition (b) of the present invention may help to support hormone balance leading up to ovulation, improves feeling of wellbeing, support a healthy libido, supports mental clarity, support thyroid health, promote iron absorption and support antioxidant capacity.

In one embodiment composition (b) of the present invention can be used as a women's health supplement.

In one embodiment composition (b) of the present invention can be a dietary or nutritional supplement.

In one embodiment the composition (b) can comprise
theacrine,
a source, form or compound of iron selected from ferrous bisglycinate chelate,
iron sulfate and combinations thereof,
potassium iodide,
maca extract.
inositol,
broccoli sprout powder, broccoli crown or broccoli extract, and
quercetin.

In one embodiment a dosage form including the composition (b) can comprise:
about 1 mg to about 500 mg of theacrine
about 0.5 mg to about 50 mg of a source, form or compound of iron,
about 0.005 mg to about 0.5 mg a source, form or compound of iodine,
about 1 mg to about 300 mg of maca,
about 5 mg to about 300 mg of inositol,
about 1 mg to about 300 mg of broccoli, and
about 1 mg to about 200 mg of quercetin.

In one embodiment a dosage form including the composition (b) can comprise:
about 1 mg to about 500 mg of theacrine,
about 0.5 mg to about 50 mg of ferrous bisglycinate chelate,
about 0.005 mg to about 0.5 mg potassium iodide,
about 1 mg to about 300 mg of maca root or maca extract,
about 5 mg to about 300 mg of inositol,
about 1 mg to about 300 mg of broccoli extract, broccoli crown or broccoli sprout powder, and
about 1 mg to about 200 mg of quercetin.

The present invention further provides a pharmaceutical composition or a dietary or nutritional supplement comprising composition (b) as described herein.

The present invention further provides a pharmaceutical composition comprising a pharmaceutically acceptable carrier, and an effective amount, for example a dosage form, of composition (b) as described herein.

The present invention further provides a dietary or nutritional supplement comprising a dietary or nutritionally acceptable carrier, and an effective amount, for example a dosage form, of composition (b) as described herein.

In one embodiment the composition (b), the dosage form including composition (b), and the pharmaceutical composition and dietary or nutritional supplement comprising composition (b) are for administration about once per day.

The present invention further provides a method of treating menstrual cycle related symptoms or a method of supporting women during the menstrual cycle comprising composition (b) as described herein.

The present invention further provides a composition (c) comprising palmitoylethanolamide (PEA), ancient peat and optionally one or more ingredients selected from apple, saffron, chasteberry, a source, form or compound of zinc, black pepper, ginger, melon or a combination thereof.

In one embodiment, the composition (c) can comprise palmitoylethanolamide (PEA), ancient peat, apple, saffron, chasteberry, a source, form or compound of zinc, black pepper, ginger and melon.

In one embodiment a dosage form including the composition (c) can comprise:
about 150 mg to about 500 mg of palmitoylethanolamide (PEA), and
about 10 mg to about 300 mg of ancient peat,
   one or more of ingredients selected from:
about 1 mg to about 300 mg apple,
about 1 mg to about 100 mg of saffron,
about 1 mg to about 100 mg of chasteberry or an extract thereof,
about 1 mg to about 150 mg of a source, form or compound of zinc,
about 0.5 mg to about 50 mg of black pepper.
about 1 mg to about 80 mg of ginger, and
about 0.5 mg to about 100 mg of melon.

In one embodiment a dosage form including the composition (c) can comprise:
about 150 mg to about 500 mg of palmitoylethanolamide (PEA), and
about 10 mg to about 300 mg of ancient peat or an extract thereof,
   one or more of ingredients selected from:
about 1 mg to about 300 mg apple extract or an extract thereof,
about 1 mg to about 100 mg of saffron or an extract thereof,
about 1 mg to about 100 mg of chasteberry or an extract thereof,
about 1 mg to about 150 mg of a source, form or compound of zinc selected from zinc gluconate, zinc oxide and combinations thereof,
about 0.5 mg to about 50 mg of black pepper or an extract thereof,
about 1 mg to about 80 mg of ginger or an extract thereof, and
about 0.5 mg to about 100 mg of melon.

In one embodiment composition (c) is an oral composition or is orally administered.

In one embodiment composition (c) is for administration to a woman during the luteal phase of the menstrual cycle.

In one embodiment composition (c) can be used for treating, improving or lessening symptoms associated with the luteal phase of the menstrual cycle.

In one embodiment composition (c) can be used for treating at least one symptom associated with the luteal phase of the menstrual cycle. Composition (c) of the present invention may be used for treating symptoms associated with the luteal phase of the menstrual cycle, selected from the group consisting of, but are not limited to hormone imbalance, hormonal decline, premenstrual tension, and premenstrual syndrome (PMS).

In one embodiment composition (c) can be used for improving or restoring healthy inflammatory responses and lessening PMS symptoms such as mild mood disturbances, mild abdominal cramps, and bloating.

Composition (c) of the present invention may help to relieve mild discomfort associated with PMS, enhance the absorption nutrients before menstruation, support a healthy inflammatory response, support the endocannabinoid system, promotes clear looking skin, reduce occasional stress, support energy levels, supports antioxidant capacity.

In one embodiment composition (c) can be used as a women's health supplement.

In one embodiment composition (c) can be a dietary or nutritional supplement

In one embodiment the composition (c) can comprise
palmitoylethanolamide,
ancient peat or an extract thereof,
apple extract or apple polyphenols,
saffron extract,
chasteberry extract.
a source, form or compound of zinc selected from zinc gluconate, zinc oxide, and combinations thereof,
black pepper extract,
ginger extract, and
melon concentrate.

In one embodiment a dosage form including the composition (c) can comprise:
about 150 mg to about 500 mg of palmitoylethanolamide (PEA),
about 10 mg to about 300 mg of ancient peat,
about 1 mg to about 300 mg apple,
about 1 mg to about 100 mg of saffron,
about 1 mg to about 100 mg of chasteberry,
about 1 mg to about 150 mg of a source, form or compound of zin,
about 0.5 mg to about 50 mg of black pepper,
about 1 mg to about 80 mg of ginger, and
about 0.5 mg to about 100 mg of melon or an extract thereof.

In one embodiment a dosage form including the composition (c) can comprise:
about 150 mg to about 500 mg of palmitoylethanolamide (PEA)
about 1 mg to about 300 mg of ancient peat,
about 1 mg to about 300 mg of apple extract or apple polyphenols.
about 1 mg to about 100 mg of saffron extract,
about 1 mg to about 100 mg of chasteberry extract,
about 1 mg to about 150 mg of a source, form or compound of zinc selected from zinc gluconate, zinc oxide and combinations thereof,
about 0.5 mg to about 50 mg of black pepper extract,
about 1 mg to about 80 mg of ginger root extract, and
about 0.5 mg to about 100 mg of melon concentrated.

The present invention further provides a pharmaceutical composition or a dietary or nutritional supplement comprising composition (c) as described herein.

The present invention further provides a pharmaceutical composition comprising a pharmaceutically acceptable carrier, and an effective amount, for example a dosage form, of composition (c) as described herein.

The present invention further provides a dietary or nutritional supplement comprising a dietary or nutritionally acceptable carrier, and an effective amount, for example a dosage form, of composition (c) as described herein.

In one embodiment the composition (c), a dosage form including composition (c), and the pharmaceutical composition and dietary or nutritional supplement comprising composition (c) are for administration once per day.

The present invention further provides a method of treating menstrual cycle related symptoms or a method of supporting women during the menstrual cycle comprising composition (c) as described herein.

The first composition (a) and/or the second composition (b) and/or the third composition (c) as described herein can further comprise inositol, lutein, L-carnitine, ascorbic acid, niacinamide, fennel, riboflavin, vitamin E, lycopene, methylcobalamin or combinations thereof.

The present invention also provides a pharmaceutical composition or a dietary or nutritional supplement comprising composition (a) as described herein and/or a composition (b) as described herein and/or a composition (c) as described herein.

Compositions (a). (b) and (c) of the present invention and their use in the methods, kits, products and compositions of the present invention are further defined as following:

### Composition (a):

Clove as used herein can comprise an extract thereof, a power thereof, or a derivative thereof. Clove as used herein can be selected from clove flower bud, clove extract, a compound extracted from Syzygium aromaticum flower buds and combinations thereof. Clove can be a compound, agent or drug extracted from cloves using ethanol or methanol extraction techniques. Clove can be a compound, agent or drug extracted from cloves using a water extraction and no carrier.

In one embodiment, a dosage form of composition (a) can comprise clove as described herein, in an amount between about 100 mg and about 300 mg, or between about 100 mg and about 250 mg, or between about 100 mg and about 200 mg, or between about 150 mg and about 300 mg, or between about 150 mg and about 250 mg, or between about 150 mg and about 200 mg.

Immortelle as used herein can comprise an extract thereof, a power thereof, or a derivative thereof. Immortelle as used herein may be selected from immortelle extract, immortelle flowering top or head, a compound extracted from Helichrysum italicum flowering tops or heads and combinations therefore. Immortelle is also known as Helichrysum Immortelle or Everlasting. Immortelle can be a compound, agent or drug extracted from immortelle flower or Everlasting flower using a water extraction and no carrier.

In one embodiment, a dosage form of composition (a) can comprise immortelle as described herein, in an amount between about 100 mg and about 300 mg, or between about 100 mg and about 250 mg, or between about 100 mg and about 200 mg, or between about 150 mg and about 300 mg, or between about 150 mg and about 250 mg, or between about 150 mg and about 200 mg.

In one embodiment, a dosage form of composition (a) may comprise clove and immortelle as described herein, in an amount between about 200 mg and about 600 mg. or between about 200 mg and about 550 mg, or between about 250 mg and about 500 mg, or between about 250 mg and about 400 mg or between about 300 mg and about 400 mg.

In one embodiment, composition (a) can include a combination of clove and immortelle, as described herein, in a 1:1 ratio.

In one embodiment, composition (a) can include a combination of clove and Immortelle which is commercially available under the trademark Hepure®.

Coenzyme Q10 (CoQ10) may be naturally found in fish or meat, soybean oil, olive oil, grapeseed oil, sunflower oil, argan oil, rice bran, and kaneka (yeast). CoQ10 may assist in energy production and help maintain optimal cardiovascular and cognitive health. CoQ10 is also a powerful oil-soluble antioxidant.

In one embodiment, a dosage form of the composition (a) can comprise CoQ10 in an amount between about 1 mg and about 50 mg, or between about 1 mg and about 40 mg, or between about 1 mg and about 30 mg, or between about 10 mg and about 50 mg, or between about 10 mg and about 40 mg, or between about 10 mg and about 30 mg, or between about 15 mg and about 50 mg, or between about 15 mg and about 40 mg, or between about 15 mg and about 30 mg, or between about 20 mg and about 30 mg, or between about 23 mg and about 27 mg.

Peppermint as used herein can comprise an extract thereof, a power thereof, or a derivative thereof. Peppermint can be selected from peppermint extract, a compound, agent or drug extracted from Mentha piperita leaves, peppermint leaf extract or combinations thereof.

In one embodiment, a dosage form of composition (a) can comprise peppermint as described herein in an amount between about 1 mg and about 80 mg, or between about 1 mg and about 60 mg, or between about 10 mg and about 80 mg, or between about 10 mg and about 60 mg, or between about 20 mg and about 80 mg. or between about 20 mg and about 60 mg, or between about 30 mg and about 80 mg, or between about 30 mg and about 60 mg, or between about 40 mg and about 80 mg, or between about 40 mg and about 60 mg or between about 50 mg and about 60 mg, or between about 52 mg and about 58 mg.

Ginger as used herein can comprise an extract thereof, a power thereof, or a derivative thereof. Ginger can be selected from ginger root extract, ginger rhizome extract, a compound extracted from Zingiber officinale rhizome or combinations thereof. In one embodiment, the root is used. Ginger can be a compound, agent or drug extracted from ginger using ethanol or methanol extraction techniques. Ginger contributes to physical well-being. In addition, ginger includes anti-inflammatory agents- and has an inhibitory effect on the synthesis of prostaglandin which helps reduce hypermenorrhea.

In one embodiment, a dosage form of composition (a) can comprise ginger as described herein in an amount between about 1 mg and about 80 mg, or between about 1 mg and about 60 mg, or between about 10 mg and about 80 mg, or between about 10 mg and about 60 mg, or between about 20 mg and about 80 mg, or between about 20 mg and about 60 mg, or between about 30 mg and about 80 mg, or between about 30 mg and about 60 mg, or between about 40 mg and about 80 mg, or between about 40 mg and about 60 mg, or between about 45 mg and about 55 mg.

Dong quai as used herein can comprise an extract thereof, a power thereof, or a derivative thereof. Dong quai as used herein can be selected from Dong quai extract, Dong quai root extract, a compound, agent or drug extracted from Angelica sinensis (Oliv.) Diels roots and combinations thereof.

In one embodiment, a dosage form of composition (a) can comprise Dong quai as described herein in an amount between about 1 mg and about 100 mg, or between about 1 mg and about 80 mg, or between about 1 mg and about 60 mg, or between about 10 mg and about 80 mg, or between about 10 mg and about 60 mg, or between about 20 mg and about 100 mg, or between about 20 mg and about 80 mg, or between about 20 mg and about 60 mg, or between about 30 mg and about 100 mg, or between about 30 mg and about 80 mg, or between about 30 mg and about 60 mg, or between about 40 mg and about 80 mg, or between about 40 mg and about 60 mg, or between about 45 mg and about 55 mg.

Cranberry as used herein can comprise an extract thereof. a power thereof, or a derivative thereof. Cranberry as used herein can be selected from cranberry extract, cranberry pomace, cranberry fiber, cranberry juice concentrate, cranberry fruit extract, a compound, agent or drug extracted from Vaccinium macrocarpon fruit and combinations thereof. Cranberries may be utilized as the plant material source, the composition can be a cranberry fiber product containing some of the bio-active values of cranberries.

In one embodiment, a dosage form of composition (a) can comprise cranberry as described herein in an amount between about 1 mg and about 90 mg, or between about 1 mg and about 70 mg, or between about 1 mg and about 50 mg, or between about 10 mg and about 90 mg, or between about 10 mg and about 70 mg, or between about 10 mg and about 50 mg, or between about 20 mg and about 90 mg, or between about 20 mg and about 60 mg, or between about 30 mg and about 90 mg, or between about 30 mg and about 70 mg, or between about 40 mg and about 90 mg, or between about 40 mg and about 70 mg, or between about 50 mg and about 70 mg, or between about 55 mg and about 65 mg.

Sage as used herein can comprise an extract thereof, a power thereof, or a derivative thereof. Sage can be selected from a sage aerial parts extract, a Salvia officinalis extract, Ursolia®, a compound, agent or drug extracted from Salvia officinalis and its aerial parts or leaves, and combinations thereof.

In one embodiment, a dosage form of composition (a) can comprise sage as described herein in an amount between about 1 mg and about 80 mg, or between about 1 mg and about 60 mg, or between about 1 mg and about 50 mg, or between about 10 mg and about 80 mg. or between about 10 mg and about 60 mg, or between about 20 mg and about 80 mg. or between about 20 mg and about 60 mg, or between about 30 mg and about 80 mg. or between about 30 mg and about 60 mg, or between about 40 mg and about 80 mg. or between about 40 mg and about 60 mg, or between about 45 mg and about 55 mg.

Niacinamide, also called nicotinamide, is one of the two major forms of niacin (vitamin B3). Niacinamide is critical in the production of energy. Other forms of vitamin B3 can be use instead or in addition to niacinamide, for example, composition (a) of the present invention and its use in the methods, kits, products and compositions of the present invention can comprise clove, immortelle, CoQ10, peppermint, ginger, a source, form or compound of copper, Dong quai, cranberry, sage, one or more forms of vitamin B such as niacinamide, niacin (nicotinic acid), or combinations thereof, a source, form or compound of magnesium and a source, form or compound of potassium.

In one embodiment, a dosage form of the composition (a) can comprise niacinamide in an amount between about 1 mg and about 50 mg, or between about 1 mg and about 40 mg, or between about 1 mg and about 30 mg, or between about 10 mg and about 50 mg, or between about 10 mg and about 40 mg, or between about 10 mg and about 30 mg, or between about 15 mg and about 50 mg, or between about 15 mg and about 40 mg, or between about 15 mg and about 30 mg, or between about 20 mg and about 30 mg, or between about 23 mg and about 27 mg.

Suitable sources, forms and compounds of copper comprise copper sulfate, copper amino acid chelate, copper gluconate, copper oxide, cupric acetate, cupric butyrate, cupric subcarbonate, cupric chloride, cupric citrate, cupric formate, cupric gluconate, cupric glycinate, cupric hydroxide, cupric oxide, cupric stearate, cupric sulfate, cupric subsulfate, copper salicylate, copper proteinate, cupric tyrosinate, copper glycerophosphate, copper picolinate and combinations thereof. In one embodiment the methods, compositions, uses, products and kits of the present invention can comprise copper gluconate. Copper is beneficial in optimizing the production of serotonin, the mineral element that serves as a co-factor in the enzymatic reaction involving tryptophan hydroxylase.

In one embodiment, a dosage form of composition (a) can comprise copper as described herein in an amount between about 0.01 mg and 20 mg, or between about 0.01 mg and 15 mg, or between about 0.01 mg and 10 mg, or between about 0.01 mg and 5 mg, or between about 0.01 mg and 1 mg, or between about 0.05 mg and 20 mg, or between about 0.05 mg and 10 mg, or between about 0.05 mg and 5 mg, or between about 0.05 mg and 1 mg, or between about 0.1 mg and 10 mg, or between about 0.1 mg and 5 mg, or between about 0.1 mg and 1 mg, or between about 0.1 mg and 0.6 mg, or between 0.1 mg and 0.4 mg, or between 0.15 mg and 0.25 mg.

Suitable sources, forms and compounds of magnesium comprise magnesium chloride. magnesium sulfate, magnesium carbonate, magnesium oxide, magnesium citrate, magnesium acetate, magnesium gluconate, magnesium aspartate, magnesium nitrate, magnesium silicate aluminate, magnesium silicate, magnesium benzoate, di-basic magnesium citrate, magnesium glycinate, magnesium formate, magnesium hydroxide, magnesium iodide, and combinations thereof. A suitable form of magnesium can include elemental magnesium. In one embodiment the methods, compositions, uses, products and kits of the present invention can comprise magnesium oxide.

In one embodiment, a dosage form of composition (a) can comprise magnesium as described herein in an amount between about 1 mg and about 100 mg, or between about 1 mg and about 80 mg. or between about 1 mg and about 50 mg, or between about 1 mg and about 40 mg, or between about 1 mg and about 20 mg, or between about 5 mg and about 100 mg, or between about 5 mg and about 80 mg, or between about 5 mg and about 60 mg, or between about 5 mg and about 50 mg, or between about 10 mg and about 50 mg, or between about 10 mg and about 40 mg, or between about 10 mg and about 30 mg.

Suitable sources, forms and compounds of potassium comprise potassium chloride, potassium acetate, potassium bicarbonate, potassium phosphate dibasic (or dipotassium hydrogenphosphate), potassium carbonate, potassium citrate, potassium gluconate, potassium glycerophosphate, potassium hydroxide, potassium nitrate, dipotassium phosphate, potassium dihydrogen phosphate, tripotassium phosphate, potassium sorbate, potassium sulfate, potassium proteinate, potassium amino acid chelate, potassium fumarate, potassium alpha ketoglutarate, potassium malate, potassium ascorbate, potassium succinate, potassium aspartate, potassium/magnesium aspartate, potassium picolinate and combinations thereof. Potassium has a diuretic effect and has the action of excreting sodium and removing swelling. In one embodiment the methods, compositions, uses, products and kits of the present invention can comprise potassium phosphate.

In one embodiment, a dosage form of composition (a) can comprise potassium as described herein in an amount between about 1 mg and about 100 mg, or between about 1 mg and about 80 mg, or between about 1 mg and about 50 mg, or between about 1 mg and about 40 mg, or between about 1 mg and about 20 mg, or between about 5 mg and about 100 mg. or between about 5 mg and about 80 mg, or between about 5 mg and about 60 mg, or between about 5 mg and about 40 mg, or between about 5 mg and about 20 mg, or between about 10 mg and about 20 mg, or between about 12 mg and about 18 mg.

### Composition (b)

1,3,7,9 tetramethyluric acid is known as theacrine. Theacrine can be extracted from kucha tea, genus Camellia (e.g. Camellia assamica var. kucha), the fruit cupuaçu, and other plants related to coffee and cacao (genera Coffea and Theobroma), such as Coffea liberica, Coffea dewevrei. Coffea abeokutae and Theobroma grandiflorum. Theacrine may be, either naturally or synthetically produced. Theacrine may be a theacrine analog including, but are not limited to, caffeine, methyl caffeine, theobromine, theophylline, liberine and methylliberine, and their variants. Theacrine is also commercially available under the trademark TeaCrine®

In one embodiment, a dosage form of composition (b) can comprise theacrine as described herein in an amount between about 1 mg and about 500 mg, between about 1 mg and about 400 mg, or between about 1 mg and about 300 mg, or between about 50 mg and about 500 mg, or between about 50 mg and about 400 mg, or between about 50 mg and about 300 mg, or between about 100 mg and about 500 mg, or between about 100 mg and about 400 mg, or between 100 mg and about 300 mg, or between 150 mg and about 250 mg, or between about 180 mg and about 220 mg.

Suitable sources, forms and compounds of iron can comprise iron complexes or any of the iron II (ferrous) or iron III (ferric) supplements, such as ferrous sulfate, ferrous chloride, ferrous gluconate, ferrous lactate, ferrous tartrate, iron-sugar-carboxylate complexes, ferrous fumarate, ferrous succinate, ferrous glutamate, ferrous citrate, ferrous pyrophosphate, ferrous cholinisocitrate, ferrous bisglycinate chelate, ferrous carbonate, ferrous iodide, ferrous oxide, ferrous succinate, ferrous ascorbate, iron 45 amino acid chelate, ferrous aspartate, ferrous peptonate, iron proteinate, iron chloride, iron reduced, iron oxide saccharated, ferric glycerophosphate, iron picolinate and combinations thereof. In one embodiment the methods, compositions, uses products and kits of the present invention can comprise ferrous bisglycinate chelate. Iron contributes to the normal formation of red blood cells and hemoglobin, to compensate for the losses of iron due to menstruation. In addition, iron helps reduce fatigue and asthenia.

In one embodiment, a dosage form of composition (b) can comprise iron as described herein in an amount between about 0.5 mg and about 50 mg, or between about 0.5 mg and about 40 mg, or between about 0.5 mg and about 30 mg, or between about 0.5 mg and about 20 mg, or between about 1 mg and about 50 mg, or between about 1 mg and about 40 mg, or between about 1 mg and about 30 mg, or between about 1 mg and about 20 mg, or between about 1 mg and about 10 mg, or between about 3 mg and about 10 mg, or between about 4 mg and about 6 mg.

Suitable sources, forms and compounds of iodine can comprise potassium iodide, sodium iodide. iodinated glycerol, kelp. Atlantic kelp, Pacific kelp, iodine-resublimed. potassium iodate, ferrous iodide, magnesium iodide, manganese iodide, iodine picolinate, iodine aspartate, iodine glycerophosphate, iodine (I₂) sodium iodate or combinations thereof. In one embodiment the methods, compositions, uses, products and kits of the present invention can comprise potassium iodide.

In one embodiment, a dosage form of composition (b) can comprise iodine as described herein in an amount between about 0.005 mg and about 0.5 mg, or between about 0.005 mg and about 0.4 mg, or between about 0.005 mg and about 0.3 mg, or between about 0.01 mg and about 0.5 mg, or between about 0.01 mg and about 0.3 mg, or between about 0.05 mg and about 0.5 mg, or between about 0.05 mg and about 0.3 mg, or between about 0.08 mg and about 0.5 mg, or between about 0.08 mg and about 0.3 mg, or between about 0.1 mg and about 0.3 mg, or between about 0.1 mg and about 0.2 mg, or between about 0.12 mg and about 0.16 mg.

Maca as used herein can comprise an extract thereof, a power thereof, or a derivative thereof. Maca as used herein can be a maca extract, a maca root extract, or a compound, agent or drug extracted from Lepidium meyenii, also called Peruvian Ginseng.

In one embodiment, a dosage form of composition (b) can comprise maca as described herein in an amount between about 1 mg and about 300 mg, or between about 1 mg and about 200 mg, or between about 1 mg and about 150 mg, or between about 10 mg and about 300 mg, or between about 10 mg and about 200 mg, or between about 10 mg and about 150 mg, or between about 20 mg and about 300 mg, or between about 20 mg and about 200 mg. between about 20 mg and about 150 mg. or between about 20 mg and about 100 mg, between about 40 mg and about 150 mg, or between about 60 mg and about 120 mg, or between about 80 mg and about 120 mg, or between about 90 mg and about 110 mg.

Inositol is a chemical compound with formula C₆H₂O₆, is a carbohydrate with a different structure than common sugars. It exists in nine isomers, in which the most important form, widely occurring in nature, is cis-1,2,3,5-trans-4,6-cyclohexanehexol or myo-inositol. Inositol can be selected from D-chiro-inositol, myo-inositol and combinations thereof.

In one embodiment, a dosage form of composition (b) can comprise inositol as described herein in an amount between about 5 mg and about 300 mg, or between about 5 mg and about 200 mg, or between about 10 mg and about 300 mg, or between about 10 mg and about 200 mg, or between about 50 mg and about 300 mg, or between about 50 mg and about 200 mg, or between about 50 mg and about 150 mg, or between about 100 mg and about 300 mg, or between about 100 mg and about 200 mg, or between about 125 mg and about 175 mg, or between about 140 mg and about 160 mg.

Broccoli as used herein can comprise an extract thereof, a power thereof, or a derivative thereof. Broccoli can be selected from broccoli extract, broccoli sprout powder, broccoli sprout extract, a compound, agent or drug extracted from Brassica oleracea L. crowns and combinations thereof. The term broccoli sprouts refer to new growth from seed germination, for example, a plant that is a few (e.g., three to four) days old.

In one embodiment, a dosage form of composition (b) can comprise broccoli as described herein in an amount between about 1 mg and about 300 mg, or between about 1 mg and about 200 mg, or between about 1 mg and about 150 mg, or between about 10 mg and about 300 mg, or between about 10 mg and about 200 mg, or between about 20 mg and about 300 mg, or between about 20 mg and about 200 mg, or between about 1 mg and about 150 mg, or between about 10 mg and about 150 mg, between about 20 mg and about 150 mg, or between about 50 mg and about 150 mg, or between about 80 mg and about 120 mg, or between about 90 mg and about 110 mg.

Quercetin is a flavonoid and can be extracted from Sophora japonica flower buds or can be Quercetin dihydrate. The source of quercetin may be selected from any natural source or of synthetic origin.

In one embodiment, a dosage form of composition (b) can comprise quercetin as described herein in an amount between about 1 mg and about 200 mg, 1 mg and about 150 mg, or between about 10 mg and about 200 mg, or between about 10 mg and about 150 mg, or between about 20 mg and about 200 mg, or between about 1 mg and about 150 mg, or between about 10 mg and about 150 mg, between about 20 mg and about 150 mg, or between 50 mg and about 150 mg, or between about 80 mg and about 120 mg, or between about 90 mg and about 110 mg.

### Composition (c)

Palmitoylethanolamide (or palmitic acid monoethanolamide or PEA) is an endocannabinoid-like lipid mediator with documented anti-inflammatory, analgesic, antimicrobial, immunomodulatory and neuroprotective effects. The source of palmitoylethanolamide (PEA) can be selected from any natural source or of synthetic origin. In one embodiment, palmitoylethanolamide (PEA) can comprise commercially available Levagen® and Levagen®+. Palmitoylethanolamide (PEA) can be in non-micronized form (non-micronized PEA), or in micronized form (PEA-m), or in ultra-micronized form (PEA-um).

In one embodiment, a dosage form of the composition (c) may comprise Palmitoylethanolamide (PEA) as described herein, in an amount between about 150 mg and about 500 mg, or about 150 mg and about 450 mg, about 150 mg and about 400 mg, or about 200 mg and about 500 mg, or between about 200 mg and about 450 mg, or about 200 mg and about 400 mg, or between about 250 mg and about 500 mg, or between about 250 mg and about 400 mg, or between about 300 mg and about 400 mg, or between about 300 mg and about 375 mg.

Other ethanolamides can be use instead or in addition to palmitoylethanolamide, for example, composition (c) of the present invention and its use in the methods, kits, products and compositions of the present invention can comprise ethanolamides selected from palmitoylethanolamide (PEA), Linolenoyl ethanolamide or combinations thereof, ancient peat, apple. saffron, chasteberry. a source, form or compound of zinc. black pepper, ginger, melon or a combination thereof.

Ancient peat as used herein can comprise an extract thereof, a power thereof, or a derivative thereof. Ancient peat (fossilized plants) may be mined from natural sources ancient peat-based bioinorganic material. Humic acids and/or fulvic acids can also be use instead or in addition to ancient peat, for example, composition (c) of the present invention and its use in the methods, kits, products and compositions of the present invention can comprise palmitoylethanolamide (PEA), one or more humic acids and/or fulvic acids selected from the organic components of soil, peats, brown coals. shales, compost, marine sediments and lake sediments, and optionally one or more ingredients selected from apple. saffron, chasteberry, a source, form or compound of zinc, black pepper, ginger, melon or a combination thereof.

In one embodiment, a dosage form of composition (c) can comprise ancient peat as described herein, in an amount between about 10 mg and about 300 mg, or between about 10 mg and about 250 mg, or between about 10 mg and about 200 mg, or between about 50 mg and about 300 mg, or between about 50 mg and about 250 mg, or between about 50 mg and about 200 mg.

Apple (apple fruit) as used herein can comprise an extract thereof, a power thereof, or a derivative thereof. Apple may be selected from apple extract, apple extract polyphenols, polyphenols from apple or combinations thereof.

In one embodiment, a dosage form of composition (c) can comprise apple as described herein, in an amount between about 1 mg and about 300 mg, or between about 1 mg and about 200 mg, or between about 1 mg and about 100 mg, or between about 10 mg and about 50 mg, or between about 1 mg and about 25 mg, or between about 1 mg and about 10 mg.

In one embodiment, a dosage form of composition (c) can comprise commercially available ElevATP®, which includes a combination of ancient peat and apple extract.

Saffron as used herein can comprise an extract thereof, a power thereof, or a derivative thereof. Saffron can be a saffron extract or a compound, agent or drug extracted from saffron or from the stigmas of Crocus sativus L. (Saffron) flowers.

In one embodiment, a dosage form of composition (c) can comprise saffron as described herein in an amount between about 1 mg and about 100 mg, or between about 1 mg and about 80 mg, or between about 1 mg and about 50 mg, or between about 1 mg and about 40 mg, or between about 1 mg and about 20 mg, or between about 5 mg and about 100 mg, or between about 5 mg and about 80 mg, or between about 5 mg and about 60 mg, or between about 5 mg and about 50 mg, or between about 5 mg and about 35 mg or between about 5 mg and about 30 mg, or between about 10 mg and about 50 mg, or between about 10 mg and about 30 mg.

Chasteberry as used herein can comprise an extract thereof, a power thereof, or a derivative thereof. Chasteberry can be a chasteberry extract or a compound extracted from Vitex agnus-castus fruit. Chasteberry enhances hormone balance by increasing luteinizing hormone (LH) and progesterone release and, therefore, ovulation frequency.

In one embodiment, a dosage form of composition (c) can comprise chasteberry as described herein in an amount between about 1 mg and about 100 mg, or between about 1 mg and about 80 mg, or between about 1 mg and about 50 mg, or between about 1 mg and about 40 mg. or between about 1 mg and about 20 mg, or between about 5 mg and about 100 mg, or between about 5 mg and about 80 mg, or between about 5 mg and about 60 mg, or between about 5 mg and about 50 mg, or between about 10 mg and about 50 mg, or between about 20 mg and about 40 mg, or between about 20 mg and about 35 mg.

Suitable sources, forms and compounds of zinc can comprise zinc gluconate, zinc oxide, zinc acetate, zinc benzoate, zinc caprylate, zinc carbonate, zinc chloride, zinc citrate, zinc formate, zinc lactate, zinc selenate, zinc stearate, zinc sulfate, zinc picolinate, zinc sulfate heptahydrate, zinc sulfate monohydrate, zinc succinate, zinc amino acid chelate, zinc proteinate, zinc fumarate, zinc aspartate, zinc ascorbate, zinc glycerophosphate and combinations thereof. A suitable form of zinc can include elemental zinc. In one embodiment the methods, compositions, uses products and kits of the present invention can comprise zinc gluconate, zinc oxide or combinations thereof.

In one embodiment, a dosage form of composition (c) can comprise zinc as described herein in an amount between about 1 mg and about 150 mg, or between about 1 mg and about 100 mg, or between about 5 mg and about 150 mg, or between about 5 mg and about 100 mg, or between about 10 mg and about 150 mg, or between about 10 mg and about 100 mg, or between about 20 mg and about 150 mg, or between about 20 mg and about 100 mg, or between about 30 mg and about 150 mg, or between about 30 mg and about 100 mg.

Black pepper as used herein can comprise an extract thereof, a power thereof, or a derivative thereof. Black pepper can be a black pepper extract, black pepper fruit extract or a compound, agent or drug extracted from Piper nigrum, such as piperine. Piperine can be commercially available under the trademark Bioperine®. The black pepper can be a compound, agent or drug extracted from black pepper using ethanol or methanol extraction techniques.

In one embodiment, a dosage form of composition (c) can comprise black pepper as described herein in an amount between about 0.5 mg and about 50 mg, or between about 0.5 mg and about 40 mg, or between about 0.5 mg and about 30 mg, or between about 0.5 mg and about 20 mg, or between about 1 mg and about 50 mg, between about 1 mg and about 40 mg, or between about 1 mg and about 30 mg, between about 1 mg and about 20 mg, or between about 1 mg and about 10 mg, or between about 2 mg and about 8 mg, or between about 4 mg and about 6 mg.

In one embodiment, a dosage form of composition (c) can comprise ginger as described herein in an amount between about 1 mg and about 80 mg, or between about 1 mg and about 60 mg. or between about 10 mg and about 80 mg, or between about 10 mg and about 60 mg. or between about 20 mg and about 80 mg, or between about 20 mg and about 60 mg. or between about 30 mg and about 80 mg, or between about 20 mg and about 60 mg. or between about 30 mg and about 80 mg, or between about 30 mg and about 60 mg. or between about 40 mg and about 60 mg, or between about 45 mg and about 55 mg.

Melon as used herein can comprise an extract thereof, a power thereof, or a derivative thereof. Melon can be a compound, agent or active extracted from Cucumis Melo L., a Cucumis melo protein extract. a melon concentrate, a concentrate of melon's bioactive molecules, the pulp from a hybrid Cucumis melo derived from the cell line 95LS444 (described in international application WO 92/02622), the active plant extract of Cucumis Melo obtained from the company BIONOV (EXTRAMEL®) or combinations thereof. The active extract of Cucumis Melo L. can be obtained by the method described in patent application FR 2 716 884.

In one embodiment, a dosage form of the composition (c) may comprise melon as described herein, in an amount between about 0.5 mg and about 100 mg, or between about 0.5 mg and about 80 mg, or between about 0.5 mg and about 50 mg, or between about 0.5 mg and about 40 mg, or between about 0.5 mg and about 20 mg, or between about 1 mg and about 100 mg, or between about 1 mg and about 80 mg, or between about 1 mg and about 60 mg, or between about 1 mg and about 50 mg, or between about 1 mg and about 30 mg, or between about 1 mg and about 20 mg.

In the uses, methods, kits, products and compositions comprising compositions (a), (b) and (c) as described herein, said compositions can be provided in any suitable dosage form known in the art. Compositions (a), (b) and (c) of the present invention can be made in a variety of forms, including a pill, a tablet, a gummy, a gel, a caplet, a tablet (such as a chewable tablet, a quick dissolve tablet or an effervescent tablet), a capsule (e.g. hard gelatin capsule or a soft gelatin capsule), soft-chew, a gel cap, a powder, pellets, granules, beads, a liquid suspension or drinkable solutions, a food product, and any other form in the context of the present invention or a combination thereof. One of ordinary skill in the art would recognize there are also other viable ways for delivering the nutritional supplement to a user. In one embodiment, the present invention provides a product kit, wherein each composition (compositions (a), (b) and (c) is in the form of a tablet, a capsule or a gummy product.

In the uses, methods, kits, products and compositions comprising compositions (a), (b) and (c) as described herein, said compositions may be administered orally in dosage forms containing conventional pharmaceutically acceptable carriers, adjuvants and vehicles.

In one embodiment the first, second and third compositions (a), (b) and (c), or dosage forms including them, as described herein can further comprise one or more carriers. adjuvants, excipients, diluents, fillers, buffers, stabilisers, preservatives, lubricants, flavoring and coloring agents or other materials well known to those skilled in the art and optionally other therapeutic or prophylactic agents.

The first, second and third compositions (a), (b) and (c), or dosage forms including them, as described herein can be in any form suitable for oral administration or use. In compositions (a), (b) and (c) as described herein, the components or ingredients can be blended with conventional excipients such as binders, including gelatin, pregelatinized starch, and the like; lubricants, such as hydrogenated vegetable oil, magnesium stearate, stearic acid, and the like; diluents, such as lactose, mannose, and sucrose; disintegrants, such as carboxymethyl cellulose and sodium starch glycolate; suspending agents, such as povidone. polyvinyl alcohol and the like; absorbents, such as silicon dioxide: preservatives, such as methylparaben, propylparaben, and sodium benzoate; surfactants, such as sodium lauryl sulfate, polysorbate 80, and the like; and colorants, such as F.D. & C dyes and lakes.

The first, second and third compositions (a), (b) and (c), or dosage forms including them, as described herein can be prepared according to any method known in the art for the manufacture of dietary supplements or nutraceutical formulations and such compositions can contain one or more agents selected from the group consisting of sweetening agents, flavoring agents, texture modifying component, edible oils, coloring agents and preserving agents to provide palatable preparations.

Tablets can contain the ingredients with non-toxic excipients that are suitable for the manufacture of tablets. These excipients can be inert diluents, such as calcium carbonate, sodium carbonate, lactose, calcium phosphate or sodium phosphate; granulating and disintegrating agents, such as corn starch, or alginic acid; binding agents, such as starch, gelatin or acacia; and lubricating agents, such as magnesium stearate, stearic acid or talc. The tablets can be uncoated or they can be coated by known techniques to delay disintegration and absorption in the gastrointestinal tract and thereby provide a sustained action over a longer period. See, for example, the processes describing microencapsulation for controlled release found in U.S. Pat. Nos. 5,846,566 and 6,022,562, which are incorporated by reference in their entirety. Other time delay material such as glyceryl monostearate or glyceryl distearate can be employed. Formulations for oral use can also be hard gelatin capsules wherein the active ingredient is mixed with an inert solid diluent, such as calcium carbonate, calcium phosphate or kaolin, or as soft gelatin capsules wherein the active ingredient is mixed with water or an oil medium, such as peanut oil, liquid paraffin or olive oil. Aqueous suspensions contain the active materials in admixture with excipients for the manufacture of aqueous suspensions. Such excipients are suspending agents, as such sodium carboxymethylcellulose, methylcellulose, hydroxypropylmethyl cellulose, sodium alginate, polyvinylpyrrolidone, gum tragacanth and gum acacia; and dispersing or wetting agents, which can be a naturally-occurring phosphatide, such as lecithin, or condensation products of an alkylene oxide with fatty acids, such as polyoxyethylene stearate, or condensation products of ethylene oxide with long chain aliphatic alcohols, such as heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, such as polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin. Natural fruits or plant juice or extracts may be used as the coloring agents, for example, purple carrot root or spirulina.

Oily suspensions can be formulated by suspending the active ingredients in an edible oil such as a vegetable oil, for example arachis oil, olive oil, sesame oil or coconut oil, or in a mineral oil such as liquid paraffin. The oily suspensions can contain a thickening agent, such as beeswax, hard paraffin or cetyl alcohol. Sweetening agents and flavoring agents may be added to provide palatable oral preparations. These compositions can be preserved by the addition of an anti-oxidant such as ascorbic acid. Dispersible powders and granules suitable for preparation of an aqueous suspension by the addition of water provide the active ingredient in admixture with a dispersing or wetting agent, suspending agent and one or more preservatives. Additional excipients, for example sweetening, flavoring and coloring agents, can also be present.

Compositions (a), (b) and (c), or the dosage forms including them, as described herein can also be in the form of oil-in-water emulsions and water-in-oil emulsions. The oily phase can be a vegetable oil, such as those described herein. Of interest are olive oil or arachis oil, or a mineral oil, such as liquid paraffin or mixtures of these. Suitable emulsifying agents can be naturally-occurring gums, such as guar gum. gum acacia or gum tragacanth; naturally-occurring phosphatides, such as soy bean, lecithin, and esters or partial esters derived from fatty acids and hexitol; anhydrides, such as sorbitan monooleate; and condensation products of the partial esters with ethylene oxide, such as polyoxyethylene sorbitan monooleate. The emulsions can also contain sweetening and flavoring agents. Syrups and elixirs may be formulated with sweetening agents, for example glycerol, propylene glycol, sorbitol or sucrose. The formulations can also contain a demulcent, a preservative and flavoring and coloring agents. This suspension can be formulated according to the known art using those suitable dispersing or wetting agents and suspending agents.

Tablets incorporating the first, second or third compositions (a), (b) and (c) as described herein can be prepared using conventional methods and materials known in the pharmaceutical art. The resulting tablets were recovered and stored for future use.

Compositions (a), (b) and (c) as described herein, can be formulated in solid form. These solid form preparations can be in the form of powders, tablets, dispersible granules, capsules, and cachets. A solid carrier is suitably one or more substances which may also act as diluents, flavoring agents, solubilizers, lubricants, suspending agents, binders or tablet disintegrating agents. The compounds of compositions (a), (b) and (c) as described herein can be mixed with the carrier having the necessary binding properties in suitable proportions and compacted to form tables in the shape and size desired. Suitable solid carriers can include, but are not limited, to magnesium carbonate, magnesium stearate, talc, sugar, lactose, pectin, dextrin, maltodextrin, starch, gelatin, tragacanth, methylcellulose, sodium carboxymethylcellulose, a low melting wax, cocoa butter, and the like.

In the uses, methods, kits, products and compositions comprising compositions (a), (b) and (c) as described herein, said compositions can be formulated in the form push-fit capsules made of gelatin as well as soft, sealed capsules made of gelatin and a plasticizer, such as glycerol or sorbitol. The push-fit capsules can contain the ingredients described herein in admixture with filler such as lactose, binders such as starches or starch-based ingredients, lubricants such as magnesium stearate and, optionally. stabilizers and other agents such as silicon dioxide.

In the uses, methods, kits, products and compositions comprising compositions (a), (b) and (c) as described herein, said compositions can also be produced as a tablet for easy intake. In one embodiment, compositions (a), (b) and (c) are formed in a tablet and bound together via a binding, for example comprising a cellulose agent, a vegetable powder (e.g. purple carrot root or spirulina) and magnesium stearate.

In the uses, methods, kits, products and compositions comprising compositions (a), (b) and (c) as described herein, said compositions can be formulates in the form of a capsule and further comprise silicon dioxide, magnesium stearate and hypromellose.

The term “subject” as used herein comprises any and all organisms and includes the term “patient.” “Subject” can refer to a human or any other animal, but preferably, refers to a female subject.

The term “extract” as used herein refers to a substance made by extracting a part of a raw material (such as, plants, fruits, blossoms, roots, herbs, seeds, vegetables, etc.). Without wishing to be bound by any theory, it is contemplated that an extraction process often involves using a solvent such as alcohols or water. Extracts may be in tinctures or in powder form.

It is understood that the embodiments of the present invention are not limited to the particular methodologies, protocols, solvents and reagents, and the like, described herein as they may vary. It is also to be understood that the terminology used herein is used for the purpose of describing particular embodiments only and not intended to limit the scope of the present invention.

It must also be noted that, as used herein and in the appended claims, the singular forms “a,” “an” and “the” include the plural reference unless the context clearly dictates otherwise. Thus, for example, a reference to “an extract” is a reference to one or more extracts and includes equivalents thereof known to those skilled in the art and so forth. The use of the term “at least one” followed by a list of one or more items (for example, “at least one of A and B”) can refer to one item selected from the listed items (A or B) or any combination of two or more of the listed items (A and B), unless otherwise indicated herein or clearly contradicted by context. The terms “comprising.” “having,” “including,” and “containing” can refer to as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless defined otherwise, all technical and scientific terms used herein have the same meaning as commonly understood by one of ordinary skill in the art to which this invention pertains. In embodiments, methods, devices and materials are described, although any methods and materials similar or equivalent to those described herein can be used in the practice or testing of the present invention. All references cited herein are incorporated by reference herein in their entirety.

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

When values are expressed as approximations, by use of the antecedent “about,” it will be understood that the particular value forms another embodiment. The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

As used herein, “treating” and like terms refer to reducing the severity and/or frequency of menstrual cycle symptoms, eliminating menstrual cycle symptoms, reducing the frequency or likelihood of menstrual cycle symptoms, and improving or remediating symptoms or discomfort caused, directly or indirectly, by the menstrual cycle.

All references including publications, patent applications, and patents cited herein are hereby incorporated by referenced to the same extent as if each reference were individually and specifically indicated to be incorporated by reference and were set forth in its entirety herein.

Embodiments of this invention are described herein, including the best mode known to the inventors for carrying out the invention. Variations of those embodiments may become apparent to those of ordinary skill in the art upon reading the foregoing description. Accordingly, this invention includes modifications and equivalents of the subject matter recited in the claims appended hereto as permitted by applicable law. Moreover, any combination of the elements described herein in variations thereof is encompassed by the invention unless otherwise indicated herein or otherwise clearly contradicted by context.

The compositions, products, uses and methods of the present invention are illustrated by the examples described herein. These examples are offered to illustrate, but not to limit the claimed invention.

### EXAMPLES

### Example 1—Composition (a)

| | Amount (mg) per |
|---|---|
| Ingredient | dosage form |
| Clove | 170 |
| Immortelle | 170 |
| CoQ10 | 25 |
| Peppermint extract | 55 |
| Ginger root extract | 50 |
| Magnesium oxide | 15.5 |
| Potassium phosphate | 14.08 |
| Copper gluconate | 0.2 |
| Dong quai | 50 |
| Cranberry extract | 60 |
| Sage aerial | 50 |
| parts extract | |
| Niacinamide | 25 |

### Example 2—Composition (b)

| | Amount (mg) per |
|---|---|
| Ingredient | dosage form |
| Theacrine | 200 |
| Ferrous bisglycinate | 5 |
| chelate | |
| Potassium iodide | 0.14 |
| Maca root | 100 |
| Inositol | 150 |
| Broccoli sprout | 100 |
| Quercetin | 100 |

### Example 3—Composition (c)

| | Amount (mg) per |
|---|---|
| Ingredient | dosage form |
| Palmitic acid | 350 |
| monoethanolamide (PEA) | |
| Ancient peat and | 150 |
| apple extract | |
| Saffron extract | 15 |
| Chasteberry extract | 30 |
| Zinc gluconate + | 36 |
| zinc oxide | |
| Black pepper extract | 5 |
| Ginger root extract | 50 |
| Melon concentrate | 10 |

### Example 4—Preparation of Compositions (a), (b) and (c) in Capsule from

A capsule comprising composition (a) was processed with the following ingredients

| | Amount (mg) |
|---|---|
| Ingredient | per capsule |
| Clove flower bud | 170.00 |
| Immortelle flowering top | 170.00 |
| Coenzyme Q10 98% ubidecarenone | 25.00 |
| Peppermint Extract | 23.65 |
| Ginger rhizome extract | 27.50 |
| Magnesium oxide 96% | 40.00 |
| Potassium phosphate dibasic | 32.00 |
| Copper gluconate 13.7% | 1.53 |
| Dong quai root extract | 50.00 |
| Cranberry Extract | 39.60 |
| Salvia officinalis extract | 37.50 |
| Niacinamide 98.5% | 29.19 |
| Maltodextrin | 65.25 |
| Silicon Dioxide | 14.10 |
| Magnesium hydroxide | 1.80 |
| Citric Acid | 0.6000 |
| Magnesium stearate | 10.0000 |
| Starch | 15.0000 |
| Hypromellose | 87.0000 |

A capsule comprising composition (b) was processed with the following ingredients:

| | Amount (mg) |
|---|---|
| Ingredient | per capsule |
| Theacrine | 200.00 |
| Ferrous bisglycinate | 19.00 |
| chelate | |
| Potassium Iodide | 0.28 |
| Maca extract | 40.00 |
| Inositol 97% | 150.00 |
| Broccoli sprout powder | 100.00 |
| Quercetin 95% | 100.00 |
| Microcrystalline | 2.52 |
| cellulose | |
| Maltodextrin | 4.87 |
| Silicon dioxide | 7.54 |
| Citric acid | 4.75 |
| Magnesium stearate | 8.50 |
| Starch | 65.81 |
| Tricalcium phosphate | 18.07 |
| Calcium silicate | 2.02 |
| Purple carrot | 2.68 |
| Hypromellose | 124.32 |

Composition (c) was processed in capsule form using silicon dioxide and magnesium stearate agents, hypromellose capsule and spirulina.

| | Amount (mg) |
|---|---|
| | per capsule |
| Palmitic acid | 315.00 |
| monoethanolamide (PEA) | |
| Ancient Peat | 148.50 |
| Apple Extract | 1.50 |
| Saffron Extract | 9.00 |
| Chaste Tree Berry | 24.00 |
| Zinc gluconate | 46.88 |
| Zinc oxide 99.0% | 42.86 |
| Bioperine, piper | 5.00 |
| nigrum ext | |
| Ginger rhizome extract | 27.50 |
| Melon Concentrate | 2.50 |
| Dextrin | 11.00 |
| Silicon Dioxide | 18.00 |
| Carnuba wax | 4.90 |
| Starch | 21.00 |
| Enzymatically fermentaed | 2.60 |
| guar bean fiber | |
| Polyglycerol | 5.25 |
| polyricinoleate | |
| Lecithin | 5.25 |
| veg /fruit oils | 15.74 |
| Vitamin E Acetate | 5.25 |
| Magnesium stearate | 18.00 |
| Hypromellose | 126.20 |
| Spirrulina | 0.80 |

### Example 5—Kit

A combination product or kit is shown in FIG. 1.

FIG. 1 shows a combination product or kit according to the invention including a blister pack and a box. The blister pack includes 4 capsules of the first composition (a) (la), 12 capsules of the second composition (b) (2) and 16 capsules of the third composition (c) (3).

The combination products or kits of the present invention can be used for different cycle lengths. The blister pack of FIG. 1 also contains 4 additional capsules of the first composition (a) (1b) which can be taken during the first fraction of the menstrual cycle, if this last more than 4 days, so the combination product or kit of the present invention is customizable depending on cycle length. The presence of 4 additional capsules of the first composition (a) (1b) also allows the same blister pack to be used for the start of a subsequent cycle if necessary.4

1. A method of treating or alleviating at least one menstrual cycle related symptom, or a method of supporting women during the menstrual cycle, the method comprising the oral administration to a subject of one, two or all, selected from:
(a) a first composition comprising clove and immortelle;
(b) a second composition comprising theacrine and broccoli; and
(c) a third composition comprising palmitoylethanolamide and ancient peat

2. A method of treating or alleviating at least one menstrual cycle related symptom, the method comprising the oral administration to a subject of one, two or all, selected from:
(a) a first composition comprising clove, immortelle, CoQ10, peppermint, ginger, a source, form or compound of copper, Dong quai, cranberry, sage, niacinamide, a source, form or compound of magnesium, a source, form or compound of potassium, or a combination thereof;
(b) a second composition comprising theacrine, a source, form or compound of iron, a source, form or compound of iodine, maca, inositol, broccoli, quercetin, or a combination thereof; and
(c) a third composition comprising palmitoylethanolamide, ancient peat, apple, saffron, chasteberry, a source, form or compound of zinc, black pepper, ginger, melon, or a combination thereof.

3. A method of supporting women during the menstrual cycle, the method comprising the oral administration to a subject of one, two or all selected from:
(a) a first composition comprising clove, immortelle, CoQ10, peppermint, ginger, a source, form or compound of copper, Dong quai, cranberry, sage, niacinamide, a source, form or compound of magnesium, a source, form or compound of potassium, or a combination thereof;
(b) a second composition comprising theacrine, a source, form or compound of iron, a source, form or compound of iodine, maca, inositol, broccoli, quercetin, or a combination thereof; and
(c) a third composition comprising palmitoylethanolamide, ancient peat, apple, saffron, chasteberry, a source, form or compound of zinc, black pepper, ginger, melon, or a combination thereof.

4. A combination product or kit comprising one, two or all selected from:
(a) a first composition comprising clove and immortelle;
(b) a second composition comprising theacrine and broccoli; and
(c) a third composition comprising palmitoylethanolamide (PEA) and ancient peat.

5. A combination product or kit comprising one, two or all selected from:
(i) a first composition (a) comprising clove, immortelle, CoQ10, peppermint, ginger, a source, form or compound of copper, Dong quai, cranberry, sage, niacinamide, a source, form or compound of magnesium, a source, form or compound of potassium, or a combination thereof;
(ii) a second composition (b) comprising theacrine, a source, form or compound of iron, a source, form or compound of iodine, maca, inositol, broccoli, quercetin or a combination thereof; and
(iii) a third composition (c) comprising palmitoylethanolamide (PEA), ancient peat, apple, saffron, chasteberry, a source, form or compound of zinc, black pepper, ginger, melon, or a combination thereof.

6. The combination product or kit according to claim 4 or 5, wherein the product or kit is used for the alleviation or treatment of at least one menstrual cycle related symptom or in a method of supporting women during the menstrual cycle.

7. Use of the combination product or kit of claim 4 or 5 for improving or lessening alterations of mood, energy levels, mood swings, mild menstrual aching, or other mild discomforts associated with the menstrual cycle.

8. Use of the combination product or kit of claim 4 or 5 as a dietary or nutritional supplement.

9. A composition comprising clove, immortelle and optionally one or more ingredients selected from CoQ10, peppermint, ginger, a source, form or compound of copper, Dong quai, cranberry, sage, niacinamide, a source, form and compound of magnesium and a source, form or compound of potassium.

10. A composition comprising clove, immortelle, CoQ10, peppermint, ginger, a source, form or compound of copper, Dong quai, cranberry, sage, niacinamide, a source, form or compound of magnesium and a source, form or compound of potassium.

11. A pharmaceutical composition or a dietary or a nutritional supplement comprising clove, immortelle and optionally one or more ingredients selected from CoQ10, peppermint, ginger, a source, form or compound of copper, Dong quai, cranberry, sage, niacinamide, a source, form and compound of magnesium and a source, form or compound of potassium.

12. A pharmaceutical composition or a dietary or a nutritional supplement comprising clove, immortelle, CoQ10, peppermint, ginger, a source, form or compound of copper, Dong quai, cranberry, sage, niacinamide, a source, form or compound of magnesium and a source, form or compound of potassium.

13. A method of alleviating or treating menstrual cycle related symptoms or a method of supporting women during the menstrual cycle, the method comprising administering the composition of claim 11 or 12 to a subject.

14. A composition comprising theacrine, and broccoli and optionally one or more ingredients selected from a source, form or compound of iron, a source, form or compound of iodine, maca, inositol and quercetin.

15. A composition comprising theacrine, a source, form or compound of iron, a source, form or compound of iodine, maca, inositol, broccoli and quercetin.

16. The composition of claim 14 or 15 for use in the alleviation or treatment of symptoms associated with the follicular phase of the menstrual cycle from the conclusion of menses or menstruation phase.

17. A pharmaceutical composition or a dietary or a nutritional supplement comprising the composition of claim 14 or 15.

18. A method of alleviating or treating menstrual cycle related symptoms or a method of supporting women during the menstrual cycle comprising administering the composition of claim 14 or 15 to a subject.

19. A composition comprising palmitoylethanolamide (PEA), ancient peat and optionally one or more ingredients selected from apple, saffron, chasteberry, a source, form or compound of zinc, black pepper, ginger and melon.

20. A composition comprising palmitoylethanolamide (PEA), ancient peat, apple, saffron, chasteberry, a source, form or compound of zinc, black pepper, ginger and melon.

21. The composition of claim 19 or 20 for use in the alleviation or treatment of symptoms associated with the luteal phase of the menstrual cycle.

22. A pharmaceutical composition or a dietary or a nutritional supplement comprising the composition of claim 19 or 20.

23. A method of alleviating or treating menstrual cycle related symptoms or a method of supporting women during the menstrual cycle, the method comprising administering the composition of claim 19 or 20 to a subject.

24. A dietary or nutritional supplement comprising the composition of claim 9 or 10, and/or the composition of claim 14 or 15, and/or the composition of claim 19 or 20.

25. The method of claims 1-3, wherein the first composition, the second composition, and the third composition is administered separately or consecutively during the menstrual cycle.

26. The combination product or kit of claim 4 or 5, wherein the product or kit is used in the alleviation of treatment of hormone imbalance, hormonal decline, premenstrual tension and premenstrual syndrome (PMS).
